(19) United States
(12) Patent Application Publication
(10) Pub. No.: US 2019/0323062 A1
(43) Pub. Date: Oct. 24, 2019

(54) STRAND SPECIFIC NUCLEIC ACID LIBRARY AND PREPARATION THEREOF

(71) Applicants: Nathalie BOLDUC, Castro Valley, CA (US); TAKARA BIO USA, INC., Mountain View, CA (US)

(72) Inventors: Nathalie BOLDUC, Castro Valley, CA (US); Marta GONZALEZ-HERNANDEZ, Ann Arbor, MI (US); Emmanuel KAMBEROV, Ann Arbor, MI (US); Brian WALSH, Brighton, MI (US)

(21) Appl. No.: 16/465,008

(22) PCT Filed: Apr. 11, 2018
(86) PCT No.: PCT/US2018/027189

§ 371 Date: May 29, 2019

Related U.S. Application Data
(60) Provisional application No. 62/484,423, filed on Apr. 12, 2017.

(51) Int. Cl. C12Q 1/6806; C12N 15/10

(52) U.S. Cl. CPC C12Q 1/6806; C12N 15/1096

(57) ABSTRACT

Provided are methods for generating strand specific nucleic acids. The subject methods may include coupling an adaptor to a 3′-end of a target nucleic acid to form an adaptor-coupled target nucleic acid, which may be combined with further components in a template switching reaction to produce a product nucleic acid. The subject methods find use in a variety of applications, including but not limited to e.g., the preparation of nucleic acid libraries.

### CROSS REFERENCE TO RELATED APPLICATIONS

Pursuant to 35 U.S.C. § 119(e), this application claims priority to the filing date of the U.S. Provisional Patent Application Ser. No. 62/484,423, filed Apr. 12, 2017; the disclosure of which application is herein incorporated by reference.

### INTRODUCTION

This section provides background information related to the present disclosure that is not necessarily prior art.

Considerable interest resides in the investigation and analyses of nucleic acids derived from various sources. In certain instances, it would be advantageous to investigate or analyze a particular nucleic acid strand, such as one strand of a nucleic acid duplex or only expressed nucleic acid strands (e.g., mRNA). Nucleic acid libraries prepared from RNA, for example, may not allow identification of the particular strand of DNA from which a particular RNA was transcribed upon sequencing thereof. Where such nucleic acid libraries are generated through the annealing of random primers, it can be difficult to determine coding nucleic acid sequence from non-coding nucleic acid sequences.

Strand specific technologies are intended to determine the particular nucleic strand from which a given RNA was transcribed. A comparison of strand specific RNA sequencing technologies can be found in Levin et al., “Comprehensive comparative analysis of strand-specific RNA sequencing methods,” Nature Methods Vol. 7, No. 9 (September 2010). Briefly, early nucleic acid strand specific protocols involved ligating adaptors to both ends of RNA fragments. These protocols required decapping, fragmentation, and dephosphorylation of mRNA prior to ligation of a 3′ adaptor. Adaptor ligated fragments would subsequently need to be phosphorylated in order to permit ligation of a 5′ adaptor. After ligating the 5′ adaptor, the adaptor flanked fragments can be reverse transcribed to produce a first strand cDNA and can be further amplified using PCR, for example.

Another method includes a stranded RNA sequencing kit provided by Illumina, Inc. (San Diego, Calif.) that also ligates adaptors to both ends of an RNA and requires uncapping and dephosphorylation, but the first ligation uses a preadenylated 3′ adaptor, which reduces the number of cleanup steps. The Illumina kit also requires the adaptor ligated fragments to be phosphorylated before the 5′ adaptor can be ligated. Following ligation of the 5′ adaptor, reverse transcription generates a first strand cDNA, which can be subsequently amplified by PCR.

The SMARTer Stranded RNA-seq kit by Takara Bio USA, Inc. (Mountain View, Calif.) uses random priming for the addition of a 3′ adaptor. Adaptor addition is followed by reverse transcription with a reverse transcriptase with template switching activity and the use of a template switch oligonucleotide. The use of template switch oligonucleotides during reverse transcription allow this method to bypass the mRNA uncapping step as well as the dephosphorylation and subsequent rephosphorylation of the target nucleic acid molecule. Following production of the first strand cDNA, PCR amplification may be performed.

Another approach described by Levin et al. includes a hybrid approach that incorporates dephosphorylation of fragmented RNA, ligation of a 3′ adaptor (without preadenylation), cleanup, and subsequent use of reverse transcription and template switching to add the remaining adaptor to the first strand cDNA.

Yet another approach includes using thermostable group II intron reverse transcriptase fusion proteins for cDNA synthesis. Briefly, these reverse transcriptase fusion proteins can template switch directly from a first template RNA to a second template RNA regardless of sequence. This activity can allow unbiased template independent attachment of a first adaptor without ligation. Reverse transcription can be followed either by circularization with CircLigase or addition of a 3′ adaptor to the cDNA via ligation.

Still another method exists in which preadenylated 3′ adaptors are ligated to fragmented RNA and reverse transcription is performed using a reverse transcription primer containing both forward and reverse Illumina sequencing platform sequences. Reverse transcription is followed by circularization of the cDNA with CircLigase. As there are primer sequences in the circularized cDNA, PCR amplification can be performed using the circularized cDNA as template. However, in order to capture the information at the 5′ end of mRNAs, they would need to be decapped.

Libraries for next generation sequencing have been prepared from ssDNA in order to sequence damaged or ancient DNA, for example. Recently, such methods have been applied to the abundance of ssDNA in cell-free fetal DNA (cfDNA). Such methods include ligating biotinylated adaptors to the 3′ end of dephosphorylated ssDNA using CircLigase II. The ligation products are subsequently immobilized on streptavidin beads and a 5′-tailed primer complementary to the adaptor is annealed and used for polymerization. The 5′ adaptor is then ligated to the blunt end joining just the 3′ end of the nascent strand and the 5′ end of the adaptor. Currently, there are a variety of commercially available technologies for sequencing double stranded nucleic acids, but very few technologies for sequencing single stranded nucleic acids.

### SUMMARY

The present technology includes systems, processes, articles of manufacture, and compositions that relate to strand specific nucleic acid libraries.

Provided are methods for generating strand specific nucleic acids. The subject methods may include coupling an adaptor to a 3′-end of a target nucleic acid to form an adaptor-coupled target nucleic acid, which may be combined with further components in a template switching reaction to produce a product nucleic acid. Product nucleic acids produced by the methods of the present disclosure will include sequence, or the complement thereof, of the target nucleic. The product nucleic acid may further include sequence, or the complement thereof, of the adaptor, the presence of which may allow for identification of the 3′-end of the target nucleic acid sequence present in the product nucleic acid. The subject methods find use in a variety of applications, including but not limited to e.g., the preparation of nucleic acid libraries.

In some embodiments, a method of the present disclosure may include coupling an adaptor to a 3′-end of a target nucleic acid to form an adaptor-coupled target nucleic acid. The method may include combining the coupled target nucleic acid with a primer comprising a 3′ domain that hybridizes to at least a portion of an adaptor domain of the coupled target nucleic, a template switch oligonucleotide, and reagents for primer extension and template switching such as e.g., a polymerase and dNTPs, into a reaction mixture. The components may be combined in the reaction mixture under conditions sufficient to produce a complex that includes the coupled target nucleic acid and the template switch oligonucleotide each hybridized to a single product nucleic acid polymerized from dNTPs in a template switching reaction in the reaction mixture.

A method of preparing a strand specific nucleic acid library is also provided. The method includes where a first adaptor is coupled to a 3′-end of a target nucleic acid to form an adaptor-coupled target nucleic acid. A primer is hybridized to the adaptor-coupled target nucleic acid. The primer is extended to form a first primer extension product complementary to a portion of the adaptor-coupled target nucleic acid. At least one non-template directed nucleotide is added to a 3′-end of the first primer extension product. A portion of a second adaptor is hybridized to the at least one non-template directed nucleotide at the 3′-end of the first primer extension product. The at least one non-template directed nucleotide at the 3′-end of the primer extension product is extended to form a second primer extension product. The resulting second primer extension product is accordingly complementary to a portion of the adaptor-coupled target nucleic acid and to a portion of the hybridized second adaptor. The method can include amplifying the second primer extension product and sequencing the amplified second primer extension product. The first adaptor can include at least one cleavable site, where the cleavable site can include uracil. Uracil can be degraded using uracil-DNA glycosylase, where the resulting abasic site can be cleaved by hydrolysis. In this manner, the at least one cleavable site in the first adaptor can be cleaved after the primer is extended to form a first primer extension product complementary to a portion of the adaptor-coupled target nucleic acid. This can minimize any aberrant effects of any remaining unligated first adaptor and can therefore minimize or eliminate subsequent purification or isolation steps.

In certain embodiments, the present technology can include ligation of a 5′ adenylated first adaptor to the 3′ end of full length or fragmented and repaired mRNA or ssDNA, which can minimize bias in the library preparation. For example, the 3′ end of various single stranded nucleic acids can have various structures that are incompatible for ligation, where such structures can be replaced by a 3′ hydroxyl group that can be ligated with first adaptor including an adenylated 5′-end. What is more, the primer for reverse transcription, a template switching reverse transcriptase or polymerase, and the second adaptor for template switching can be provided in a single reaction volume. Reverse transcription can thereby add at least one non-template directed nucleotide (e.g., 2-4 cytosines) at the 3′ end of the first primer extension product, which are then hybridized by the second adaptor, allowing the reverse transcriptase to continue primer extension through the second adaptor to form the second primer extension product. These steps bypass the need to decap mRNA used as the target nucleic acid, thereby simplifying library preparation. The second primer extension product can then be amplified by PCR. The first adaptor can additionally include multiple uracils that can be degraded and hence obviate the need for an extra cleanup step.

Various strand specific nucleic acid libraries can therefore be formed by the various methods provided herein.

Likewise, various kits for preparing a strand specific nucleic acid library can include the various components used in the methods provided herein.

Further areas of applicability will become apparent from the description provided herein. The description and specific examples in this summary are intended for purposes of illustration only and are not intended to limit the scope of the present disclosure.

### BRIEF DESCRIPTION OF THE FIGURES

The drawings described herein are for illustrative purposes only of selected embodiments and not all possible implementations, and are not intended to limit the scope of the present disclosure.

FIG. 1 illustrates a method of preparing a strand specific nucleic acid library according to a first embodiment of the present technology.

FIG. 2 illustrates a method of preparing a strand specific nucleic acid library according to a second embodiment of the present technology.

FIG. 3 provides a schematic representation generally depicting a template-switching reaction.

FIG. 4 illustrates an exemplary embodiment of the method of the disclosure.

FIG. 5 shows sequencing metrics from samples prepared by the methods of the disclosure.

FIG. 6A-6C compares sequencing coverage across low abundance transcripts at different input amounts.

FIG. 7A-7C compares sequencing coverage across high abundance transcripts at different input amounts.

FIG. 8 illustrates reproducibility of the method at different input amounts.

FIG. 9 shows sequencing metrics from samples prepared by methods of the disclosure.

FIG. 10 illustrates an exemplary embodiment of a method for sequencing small and large RNAs using the methods of the disclosure.

FIG. 11 illustrates an exemplary embodiment of a method for sequencing small and large RNAs using the random priming methods of the disclosure.

FIG. 12 illustrates an exemplary embodiment of a method for sequencing small and large RNAs using the Oligo-dT priming methods of the disclosure.

### DEFINITIONS

As used herein, the term “hybridization conditions” means conditions in which a primer, or other polynucleotide, specifically hybridizes to a region of a target nucleic acid with which the primer or other polynucleotide shares some complementarity. Whether a primer specifically hybridizes to a target nucleic acid is determined by such factors as the degree of complementarity between the polymer and the target nucleic acid and the temperature at which the hybridization occurs, which may be informed by the melting temperature (T_{M}) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a region of the product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The term “primer” as used herein, refers to an oligonucleotide which acts to initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal concentration, and salt concentration. Primers are generally of a length compatible with their use in synthesis of primer extension products, and may be in a range from 6 bp to 150 bp or more, including but not limited to e.g., 6 bp to 150 bp, 7 bp to 150 bp, 8 bp to 150 bp, 9 bp to 150 bp, 10 bp to 150 bp, 11 bp to 150 bp, 12 bp to 150 bp, 13 bp to 150 bp, 14 bp to 150 bp, 15 bp to 150 bp, 16 bp to 150 bp, 17 bp to 150 bp, 18 bp to 150 bp, 19 bp to 150 bp, 20 bp to 150 bp, 25 bp to 150 bp, 30 bp to 150 bp, 35 bp to 150 bp, 40 bp to 150 bp, 6 bp to 100 bp, 7 bp to 100 bp, 8 bp to 100 bp, 9 bp to 100 bp, 10 bp to 100 bp, 11 bp to 100 bp, 12 by to 100 bp, 13 bp to 100 bp, 14 bp to 100 bp, 15 bp to 100 bp, 16 bp to 100 bp, 17 bp to 100 bp, 18 bp to 100 bp, 19 bp to 100 bp, 20 bp to 100 bp, 25 bp to 100 bp, 30 bp to 100 bp, 35 bp to 100 bp, 40 bp to 100 bp, 6 bp to 40 bp, 7 bp to 40 bp, 8 bp to 40 bp, 9 bp to 40 bp, 10 bp to 40 bp, 11 bp to 40 bp, 12 bp to 40 bp, 13 bp to 40 bp, 14 bp to 40 bp, 15 bp to 40 bp, 16 bp to 40 bp, 17 bp to 40 bp, 18 bp to 40 bp, 19 bp to 40 bp, 20 bp to 40 bp, 25 bp to 40 bp, 30 bp to 40 bp, 35 bp to 40 bp, 6 bp to 20 bp, 7 bp to 20 bp, 8 bp to 20 bp, 9 bp to 20 bp, 10 bp to 20 bp, 11 bp to 20 bp, 12 bp to 20 bp, 13 bp to 20 bp, 14 bp to 20 bp, 15 bp to 20 bp, 16 bp to 20 bp, 17 bp to 20 bp, 18 bp to 20 bp, and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length. Primer lengths as described herein may include only the portion of the primer that hybridizes to its complementary template and may exclude, e.g., portions that are not complementary to a template including but not limited to e.g., homology arms that may not be complementary to a template.

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

A domain refers to a stretch or length of a nucleic acid made up of a plurality of nucleotides, where the stretch or length provides a defined function to the nucleic acid. Examples of domains include primer binding domains, hybridization domains, barcode domains (such as source barcode domains), unique molecular identifier (UMI) domains, Next Generation Sequencing (NGS) adaptor domains, NGS indexing domains, etc. In some instances, the terms “domain” and “region” may be used interchangeably, including e.g., where immune receptor chain domains/regions are described, such as e.g., immune receptor constant domains/regions. While the length of a given domain may vary, in some instances the length ranges from 2 to 100 nt, such as 5 to 50 nt, e.g., 5 to 30 nt.

### DETAILED DESCRIPTION

Provided are methods for generating strand specific nucleic acids. The subject methods may include coupling an adaptor to a 3′-end of a target nucleic acid to form an adaptor-coupled target nucleic acid, which may be combined with further components in a template switching reaction to produce a product nucleic acid. Product nucleic acids produced by the methods of the present disclosure will include sequence, or the complement thereof, of the target nucleic. The product nucleic acid may further include sequence, or the complement thereof, of the adaptor, the presence of which may allow for identification of the 3′-end of the target nucleic acid sequence present in the product nucleic acid. The subject methods find use in a variety of applications, including but not limited to e.g., the preparation of nucleic acid libraries.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. Except where otherwise expressly indicated, all numerical quantities in this description are to be understood as modified by the word “about” and all geometric and spatial descriptors are to be understood as modified by the word “substantially” in describing the broadest scope of the technology.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order which is logically possible.

As summarized above, the present disclosure provides methods for generating strand specific nucleic acids and libraries thereof, i.e., strand specific nucleic acid libraries. By “strand specific”, in the context of the subject nucleic acids and libraries, is generally meant that the nucleic acids and libraries are produced in a manner allowing for retrospective identification of the nucleic acid strand, and/or the ends of a nucleic acid strand, from which they were derived. For example, a strand specific product nucleic acid produced by the methods of the present disclosure may include sequence, or the complement thereof, the presence of which may allow for identification of whether the product nucleic acid was generated from a 3′ end or a 5′ end of a target nucleic acid, and/or which end of the product nucleic acid corresponds to the 3′ end and/or the 5′ end of the target nucleic acid from which it was derived. In some instances, a strand specific product nucleic acid produced by the methods of the present disclosure may include sequence, or the complement thereof, of an adaptor and the target nucleic, such that the presence and/or location of the adaptor sequence may allow for identification of which sequence present in the produced product nucleic acid corresponds to the 3′-end of the target nucleic acid from which it was derived.

Methods of the present disclosure may include coupling of an adaptor nucleic acid to a target nucleic acid in generating strand specific nucleic acids and/or libraries thereof. For example, the methods of the present disclosure may include coupling an adaptor to a 3′-end of a target nucleic acid to form an adaptor-coupled target nucleic acid. Coupling of a particular end of target nucleic acid (e.g., the 3′ end) to an adaptor, e.g., where the adaptor includes an identifiable sequence such as a barcode, may allow for strand specific identification of the target nucleic acid in later processes. As a non-limiting example, in some instances, following the production of a product nucleic acid, and/or library thereof, and subsequent sequencing the adaptor may allow for strand specific identification of the 3′ end of the target nucleic acid sequences due to the known coupling of the adaptor specifically to the 3′ end of the target nucleic acid in an earlier step.

The methods of the present disclosure are suitable for use with a variety of different target nucleic acids, described in more detail below. For example, the present methods may be employed to produce strand specific nucleic acids and/or libraries thereof, e.g., for use in sequencing, of both small and large target nucleic acids including both polyadenylated and non-polyadenylated target nucleic acids (e.g., large polyadenylated target nucleic acids, small polyadenylated target nucleic acids, large non-polyadenylated target nucleic acids, small non-polyadenylated target nucleic acids, including combinations thereof). In some instances, the methods of the present disclosure may be employed to separately produce strand specific nucleic acids and/or libraries there of small and large target nucleic acids including polyadenylated and non-polyadenylated target nucleic acids. However, the present methods are not limited to separate generation and, in some instances, may be employed to simultaneously generate strand specific product nucleic acids and/or libraries thereof of both small and large target nucleic acids. In some instances, the present methods may be employed to simultaneously generate strand specific product nucleic acids and/or libraries thereof of both polyadenylated and non-polyadenylated target nucleic acids.

In some instances, coupling of an adaptor of known sequence to a target nucleic acid in the methods of strand specific nucleic acid and/or library generation described herein allows for strand specific identification of the exact sequence of the target nucleic acid end.

A comparison with methods of nucleic acid and/or library preparation employing a poly-A-tailing reaction is illustrative. For example, in a poly-A-tailing reaction a plurality of adenosine monophosphates are added to the 3′ end of non-polyadenylated transcripts, e.g., to allow for a library to be prepared from polyadenylated transcripts and non-polyadenylated transcripts in the same reaction, e.g., through the use of a reverse transcription reaction utilizing a poly(dT) first strand primer. By polyadenylating the non-polyadenylated transcripts the poly(dT) primer can be employed to capture the sequence of the 3′ ends of the previously non-polyadenylated transcripts at the same time as the polyadenylated transcripts. However, for previously non-polyadenylated transcripts that already contained one or more adenosines at their 3′ ends prior to polyadenylation, the boundary between any previously present adenosine(s) and the added adenosines cannot be precisely known. As such, the exact sequences of the 3′ ends of non-polyadenylated transcripts cannot necessarily be determined where poly-A-tailing is employed. Put another way, using poly-A-tailing reactions, one cannot always determine if the initial target nucleic acid did or did not contain one or more terminal adenosines.

In contrast, where an adaptor of known sequence is ligated to the 3′ end of target nucleic acids (whether such target nucleic acids are polyadenylated or non-polyadenylated) as employed in embodiments of the present disclosure, the exact sequence of the 3′ ends of the target nucleic acids may be determined. For example, because the sequence of the adaptor is known and the adaptors is directly ligated to the 3′ end of the target nucleic acid, the first nucleic acid of the 3′ end may be identified by virtue of being adjacent to the last nucleotide of the known adaptor sequence. Accordingly, methods of the present disclosure may allow for the identification of the exact sequence present at the ends of target nucleic acids, including where the methods are employed to simultaneously prepare strand specific nucleic acids, and/or libraries thereof, from combinations of small, large, polyadenylated and non-polyadenylated target nucleic acids, and sub-combinations thereof.

Following coupling reactions employing the instant methods the coupled nucleic acids may be said to contain one or more domains. Domains of coupled nucleic acids may, for example, be derived from and/or defined by the individual nucleic acids that were coupled. For example, following the coupling of an adaptor and a target nucleic acid, an adaptor-coupled target nucleic acid may be said to have an “adaptor domain”, defined as the portion of the adaptor-coupled nucleic acid made up of the previously independent adaptor. Similarly, following the coupling of an adaptor and a target nucleic acid, an adaptor-coupled target nucleic acid may be said to have a “target nucleic acid domain”, defined as the portion of the adaptor-coupled nucleic acid made up of the previously independent target nucleic acid.

Domains of larger nucleic acids may be useful in, among other things, defining the interactions of portions of the nucleic acid with other components of a reaction. For example, a domain of a nucleic acid may be, or may contain a portion that is, complementary with a component of a reaction, such as, e.g., a primer, one or more non-template nucleotides, etc. Any useful section of a nucleic acid may be defined as a domain as appropriate in the methods, and descriptions thereof, herein.

In some embodiments, the present technology relates to preparation of a strand specific nucleic acid library by coupling a first adaptor to a 3′-end of a target nucleic acid to form an adaptor-coupled target nucleic acid. A primer is hybridized to the adaptor-coupled target nucleic acid. Extending the primer forms a first primer extension product complementary to a portion of the adaptor-coupled target nucleic acid. At least one non-template directed nucleotide is added to a 3′-end of the first primer extension product. Hybridizing a portion of a second adaptor to the at least one non-template directed nucleotide at the 3′-end of the first primer extension product allows the at least one non-template directed nucleotide at the 3′-end of the primer extension product to be extended and form a second primer extension product complementary to a portion of the adaptor-coupled target nucleic acid and to a portion of the hybridized second adaptor. The second primer extension product can be amplified and sequenced. In this manner, a strand specific nucleic acid library can be prepared that originates from particular target nucleic acid strands of interest, such as one strand of a nucleic acid duplex or only from an expressed nucleic acid strand (e.g., mRNA). The method may, in some instances, serve to reduce bias, minimize aberrant reaction products, maximize efficiency and representation in the library, reduce loss of material by minimizing and/or eliminating purification steps, and/or can be more cost effective than other methods.

### Template Switching

As summarized above, the present methods may include the use of a template switching reaction. For example, certain reaction components may be combined in a reaction mixture under conditions sufficient for a template switching reaction to occur. In some instances, the subject methods may include generating a product nucleic acid from a nucleic acid sample using a template switching reaction, including e.g., a template switching reverse transcription reaction.

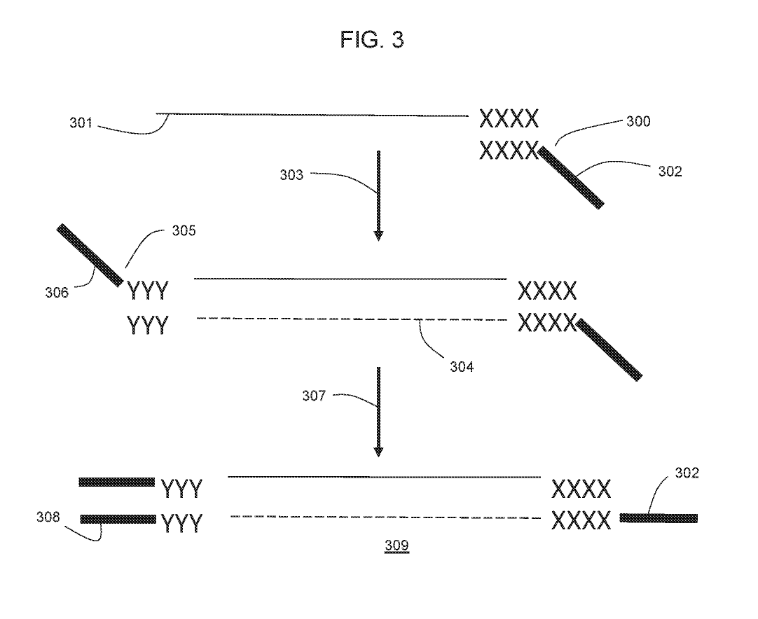

A general depiction of a template switching reaction is schematized in FIG. 3. In the example shown, a nucleic acid primer 300 hybridizes to a template nucleic acid 301 (also referred to as a “target nucleic acid” herein) through complementary sequence (represented by “XXXX”) shared by the primer and the template. The primer may, but need not necessarily, include a region of additional sequence 302 that is not complementary to the template (e.g., non-templated). Following annealing of the primer to the template, primer extension 303 proceeds, e.g., through the use of a reverse transcriptase, to generate a single product nucleic acid strand 304 (also referred to herein as an “extension product”) that is complementary to the template. The employed polymerase (e.g., reverse transcriptase), having terminal transferase activity, transfers non-templated nucleotides to the generated single product nucleic acid (represented by “YYY”) and a template switching oligonucleotide 305 (also referred to herein as a “second adaptor”) hybridizes to the non-templated nucleotides of the single product nucleic acid by a sequence of complementary nucleotides (also represented by “YYY” and also referred to herein as a 3′ hybridization domain) present on the template switch oligonucleotide. The template switch oligonucleotide includes additional sequence 306 that does not hybridize to the non-templated nucleotides. Template switching 307 occurs, wherein the polymerase (e.g., reverse transcriptase) switches from the template to utilize the template switching oligonucleotide as a second template, transcribing the additional sequence 306 to generate its complement 308. The now fully generated single product nucleic acid strand 309 includes the complete sequence of the primer, including any additional sequence 302, if present, that did not hybridize to the template, the complementary sequence of the template and the complementary sequence of the template switch oligonucleotide. Methods and reagents related to template switching are also described in U.S. Pat. No. 9,410,173; the disclosure of which is incorporated herein by reference in its entirety.

In some instances, methods of the present disclosure include combining a RNA, DNA or RNA/DNA sample (e.g., adaptor coupled-RNA, -DNA, or both), a primer, a template switch oligonucleotide, polymerase (such as a reverse transcriptase), and dNTPs, in a reaction mixture under conditions sufficient to produce a product nucleic acid including a template (or target) nucleic acid and the template switch oligonucleotide each hybridized to adjacent regions of a first strand product nucleic acid (or elongation product). One or more components of the reaction may or may not include a barcode, a sequencing adapter domain (e.g., an Illumina® Read Primer 2 sequence), a PCR primer binding domain (e.g., a domain that binds the Clontech® Primer IIA), a blocking modification, and the like.

In the first strand synthesis, the reverse transcriptase template switches from the template to a template switch oligonucleotide (e.g., a Clontech SMART-Seq v4 template switch oligonucleotide) that includes a 3′ hybridization domain that may include an LNA and a 5′ domain including, for example, a second PCR primer binding domain. In some instances, the second PCR primer binding domain (e.g., a domain that binds the Clontech® Primer IIA) is the same as the first PCR primer binding domain. In some instances, the second PCR primer binding domain is different (e.g., differs by at least one nucleotide) from the first PCR primer binding domain. After first-strand synthesis, the single product nucleic acid may be PCR amplified, e.g., using a blocked Clontech® Primer IIA to generate product nucleic acid, including e.g., product double stranded DNA, such as double stranded cDNA. This example is provided for illustrative purposes and should not be deemed to be limiting.

A template-switching reaction may make use of a suitable reaction mixture. Suitable reaction mixtures for a template-switching reaction may include the template switch oligonucleotide at a concentration sufficient to readily permit template switching of the polymerase from the template to the template switch oligonucleotide and further elongation by a polymerase as templated by any additional sequence, if present, of the template switch oligonucleotide. For example, the template switch oligonucleotide may be added to the reaction mixture at a final concentration of from 0.01 to 100 μM, such as from 0.1 to 10 μM, such as from 0.5 to 5 μM, including 1 to 2 μM (e.g., 1.2 μM).

In some instances, a polymerase combined into a template-switching reverse transcription reaction mixture is capable of template switching, where the polymerase uses a first nucleic acid strand as a template for polymerization, and then switches to the 3′ end of a second template nucleic acid strand to continue the same polymerization reaction. In some instances, the polymerase capable of template switching is a reverse transcriptase. Reverse transcriptases capable of template-switching that find use in practicing the subject methods include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants derivatives, or functional fragments thereof, e.g., RNase H minus or RNase H reduced enzymes. For example, the reverse transcriptase may be a Moloney Murine Leukemia Virus reverse transcriptase (MMLV RT) or a Bombyx mori reverse transcriptase (e.g., Bombyx mori R2 non-LTR element reverse transcriptase). Polymerases capable of template switching that find use in practicing the subject methods are commercially available and include SMARTScribe™ reverse transcriptase and PrimeScript™ reverse transcriptase available from Takara Bio USA, Inc. (Mountain View, Calif.).

A template-switching reverse transcription reaction of the present methods may include the use of a polymerase having terminal transferase activity. For example, the polymerase (e.g., a reverse transcriptase such as MMLV RT) combined into the reaction mixture has terminal transferase activity such that a homonucleotide stretch (e.g., a homo-trinucleotide, such as C—C—C) may be added to the 3′ end of a nascent strand, and the 3′ hybridization domain of the template switch oligonucleotide includes a homonucleotide stretch (e.g., a homo-trinucleotide, such as G-G-G) complementary to that of the 3′ end of the nascent strand. In other aspects, when the polymerase having terminal transferase activity adds a nucleotide stretch to the 3′ end of the nascent strand (e.g., a trinucleotide stretch), the 3′ hybridization domain of the template switch oligonucleotide includes a hetero-trinucleotide comprises a nucleotide comprising cytosine and a nucleotide comprising guanine (e.g., an r(C/G)₃ oligonucleotide), which hetero-trinucleotide stretch of the template switch oligonucleotide is complementary to the 3′ end of the nascent strand. Examples of 3′ hybridization domains and template switch oligonucleotides are further described in U.S. Pat. No. 5,962,272, the disclosure of which is herein incorporated by reference.

A polymerase with terminal transferase activity is capable of catalyzing the addition of deoxyribonucleotides to the 3′ hydroxyl terminus of a RNA or DNA molecule. In certain aspects, when the polymerase reaches the 5′ end of the template, the polymerase is capable of incorporating one or more additional nucleotides at the 3′ end of the nascent strand not encoded by the template. For example, when the polymerase has terminal transferase activity, the polymerase may be capable of incorporating 1, 2, 3, 4, 5, 6, 7, 8, 9, 10 or more additional nucleotides at the 3′ end of the nascent strand. All of the nucleotides may be the same (e.g., creating a homonucleotide stretch at the 3′ end of the nascent strand) or one or more of the nucleotides may be different from the other(s) (e.g., creating a heteronucleotide stretch at the 3′ end of the nascent strand). In certain aspects, the terminal transferase activity of the polymerase results in the addition of a homonucleotide stretch of 2, 3, 4, 5, 6, 7, 8, 9, 10 or more of the same nucleotides (e.g., all dCTP, all dGTP, all dATP, or all dTTP). For example, according to one embodiment, the polymerase is an MMLV reverse transcriptase (MMLV RT). MMLV RT incorporates additional nucleotides (predominantly dCTP, e.g., three dCTPs) at the 3′ end of the nascent strand. As described in greater detail elsewhere herein, these additional nucleotides may be useful for enabling hybridization between a 3′ hybridization domain of a template switch oligonucleotide and the 3′ end of the nascent strand, e.g., to facilitate template switching by the polymerase from the template to the template switch oligonucleotide.

Reverse transcriptase utilized in the subject methods may, in some instances, be a thermo-sensitive polymerase, i.e., a polymerase that is not thermostable. Such thermo-sensitive polymerases may become inactive at a temperature above their active temperature range. For example, in some instances, a thermos-sensitive polymerase may become inactive or demonstrate significantly reduced activity after being exposed to temperatures of 75° C. or higher, 80° C. or higher, 85° C. or higher, 90° C. or higher or 95° C. or higher.

Where a reverse transcriptase is employed, it may be combined into the reaction mixture such that the final concentration of the reverse transcriptase is sufficient to produce a desired amount of the RT reaction product, e.g., a desired amount of a single product nucleic acid. In certain aspects, the reverse transcriptase (e.g., an MMLV RT, a Bombyx mori RT, etc.) is present in the reaction mixture at a final concentration of from 0.1 to 200 units/μL (U/μL), such as from 0.5 to 100 U/μL, such as from 1 to 50 U/μL, including from 5 to 25 U/μL, e.g., 20 U/μL.

### Adaptors

As summarized above, the present methods may involve the preparation of a strand specific nucleic acid library that includes coupling a first adaptor to a 3′-end of a target nucleic acid to form an adaptor-coupled target nucleic acid.

The first adaptor can include an adenylated 5′-end and a blocked 3′-end. The blocked 3′-end can include one or more blocking groups that prevent modification of the respective terminus by one or more enzyme activities or chemical modifications. For example, the blocking group can prevent ligation at the blocked terminus, polymerase extension at the blocked terminus, or exonuclease activity at the blocked terminus.

The first adaptor can include a barcode. The barcode can include a unique sequence that can serve to identify a particular target nucleic acid molecule. As used herein, the term “barcode” can refer to a polynucleotide sequence that can be used for identification of the nucleic acid to which the barcode is attached. A barcode can be an arbitrary sequence. A barcode can comprise a defined sequence. A barcode can comprise a random sequence. A barcode can be used to identify the sample from which a target polynucleotide originated. A barcode can be used for uniquely marking molecules of interest (e.g., a unique molecular identifier (UMI), i.e., for determining expression levels based on the number of instances the unique tag is sequenced. A barcode can comprise a primer binding site. Coupling the first adaptor to the 3′-end of the target nucleic acid can include ligating the adenylated 5′-end of the first adaptor to a 3′-hydroxyl group of the target nucleic acid. Coupling of the first adaptor to the target nucleic acid can include the use of one or more various ligases, such as DNA ligase and single-strand DNA ligase, RNA ligase, and single-strand RNA ligase. Examples of RNA ligases include T4 RNA ligase 1, truncated T4 RNA ligase 2, T4 RNA ligase 2 truncated K227Q, T4 RNA ligase 2 truncated KQ, and mutant versions thereof. Where the first adaptor includes an adenylated 5′-end, truncated T4 RNA ligase 2, or a mutant form thereof, can be used to ligate the 5′-end of the first adaptor to a 3′-end of the target nucleic acid including a hydroxyl group. T4 RNA ligase 1 can be used to ligate a hydroxyl group to a 5′-end of a nucleic acid including a phosphate group.

A 3′-hydroxyl group on a target nucleic acid to which an adaptor may be coupled can be preexisting on the target nucleic acid or can be formed by reacting the target nucleic acid with a member selected from the group consisting of a kinase, a phosphatase, and combinations thereof. For example, the target nucleic acid can be the result of chemical and/or physical fragmentation and can have various chemical moieties at the ends thereof, which may include moieties that are not suitable for coupling with the first adaptor at the 3′ end and/or other processing steps or reactions. Such moieties can include the following terminal chemical groups: 5′ phosphate, 3′ hydroxyl, 5′ hydroxyl, 3′ phosphate, 2′ phosphate and 3′ hydroxyl, and 2′-3′ cyclic phosphate. Treatment of the target nucleic acid with kinase and/or phosphatase can repair the ends to leave 5′ phosphate and 3′ hydroxyl terminal chemical groups. The kinase can include T4 polynucleotide kinase (T4 PNK) and the phosphatase can include recombinant shrimp alkaline phosphatase (rSAP), for example. Treatment of the target nucleic acid can include the use of various polishing methods, including the use of T4 or Pfu polymerases.

The first adaptor can be coupled to a solid or semi-solid support. The solid or semi-sold support can be any solid or semi-sold support, including but not limited to, a bead (e.g., magnetic bead, paramagnetic bead, silica bead), an array, a glass slide, a chip (e.g., microwell chip), and a hydrogel. In some instances, a plurality of first adaptors can be coupled to a solid or semi-solid support. When a plurality of first adaptors are coupled to a solid or semi-solid support, the first adaptors can comprise one or more barcodes. For example, the first adaptors can comprise a barcode for determining sample identity and a barcode for determining expression levels (e.g., UMI). In some instances, the plurality of first adaptors coupled to the solid or semi-solid support can comprise the same barcodes for determining sample identity and different barcodes for determining expression levels (e.g., different UMIs).

Adaptors, and/or portions thereof, may be configured to be degradable. Various methods may be employed in the configuration of degradable adaptors including but not limited to e.g., the inclusion of one or more cleavable sites within the adaptor. Sites may be employed for specific degradation of the adaptor, or a portion thereof, at a desired time, e.g., through enzymatic cleavage at the cleavable site.

As summarized, the first adaptor can also include at least one cleavable site. The cleavable site can permit degradation of excess or uncoupled first adaptor, thereby minimizing or preventing aberrant reactions and reaction products. The cleavable site can include one or more labile bases that can be degraded and/or result in strand scission. For example, the first adaptor can include a plurality of cleavable sites where subsequent cleavage reduces the first adaptor to fragments with substantially no propensity to participate in any further hybridization, ligation, priming, or amplification events. Examples include where the first adaptor includes from one to ten cleavable sites. The cleavage sites can be arranged throughout the first adaptor such that the first adaptor is reduced to various combinations of oligonucleotides ranging from one to six nucleotides in length. In certain embodiments, the at least one cleavable site comprises uracil, which can be degraded using uracil-DNA glycosylase (UDG), where the resulting abasic site can be cleaved by hydrolysis to fragment the first adaptor. The at least one cleavable site can comprise a ribobase and/or a restriction endonuclease cleavage site. Where the first adaptor includes one or more cleavable sites, the one or more cleavable sites can be cleaved in the present methods after the primer is extended to form a first primer extension product complementary to a portion of the adaptor-coupled target nucleic acid. In this instance, for example, any coupled first adaptor may no longer be required for primer hybridization thereto and/or any uncoupled first adaptor can be degraded to minimize or prevent its unintended and undesired participation in future reactions or modifications. One notable advantage afforded by degradation of the first adaptor is the elimination of the need to purify or isolate the adaptor-coupled target nucleic acid and/or the first primer extension product from uncoupled first adaptor. Elimination of such intermittent purification step(s) in the present method saves considerable time and maximizes yield.

The first adaptor can include an affinity tag that can be used to isolate the adaptor-coupled target nucleic acid. Isolation of the adaptor-coupled target nucleic acid using the affinity tag can include isolating the adaptor-coupled target nucleic acid from any uncoupled first adaptor and any uncoupled target nucleic acid.

### Target Nucleic Acids

As summarized above, the methods of the present disclosure may be employed using a variety of target nucleic acids (also referred to in some instances as “template nucleic acids”). The present methods may be employed to generate strand specific nucleic acids and/or libraries thereof of the various target nucleic acids.

Useful target nucleic acids can be single-stranded or double-stranded and can include DNA, RNA, or both. The target nucleic acid can be derived from natural or synthetic sources. For example, the target nucleic acid can be derived from a single cell, a cell culture, a tissue, an organ, or an organism.

Target nucleic acids of the subject disclosure may contain a plurality of distinct target nucleic acids of differing sequence. Target nucleic acids (e.g., a target RNA, a target DNA, or the like) may be polymers of any length. While the length of the polymers may vary, in some instances the polymers are 10 nts or longer, 20 nts or longer, 50 nts or longer, 100 nts or longer, 500 nts or longer, 1000 nts or longer, 2000 nts or longer, 3000 nts or longer, 4000 nts or longer, 5000 nts or longer or more nts. In certain aspects, template nucleic acids are polymers, where the number of bases on a polymer may vary, and in some instances is 10 nts or less, 20 nts or less, 50 nts or less, 100 nts or less, 500 nts or less, 1000 nts or less, 2000 nts or less, 3000 nts or less, 4000 nts or less, or 5000 nts or less, 10,000 nts or less, 25,000 nts or less, 50,000 nts or less, 75,000 nts or less, 100,000 nts or less.

Target nucleic acids may be small nucleic acids, where the length of small nucleic acids will vary and may include, but are not limited to e.g., nucleic acids of 100 nts or less in length, including but not limited to e.g. 90 nts or less, 80 nts or less, 70 nts or less, 60 nts or less, 50 nts or less, 40 nts or less, 30 nts or less, etc. Small nucleic acids may be small DNAs and/or small RNAs, including coding and non-coding DNAs and/or RNAs. Target nucleic acids can comprise a polyA tail. Target nucleic acids can comprise a cap structure.

Target nucleic acids may be large nucleic acids, where the length of large nucleic acids will vary and may include, but are not limited to e.g., nucleic acids of 150 nts or greater in length, including but not limited to e.g. 200 nts or greater, 250 nts or greater, 300 nts or greater, 350 nts or greater, 400 nts or greater, 500 nts or greater, 600 nts or greater, 700 nts or greater, 800 nts or greater, 900 nts or greater, 1000 nts or greater, etc. Large nucleic acids may be large DNAs and/or large RNAs, including coding and non-coding DNAs and/or RNAs.

In some instance, small and large target nucleic acids may be present together in a sample. The methods of the present disclosure may be employed to generate strand specific nucleic acids and/or libraries thereof of small and large nucleic acids separately or together, including where the strand specific nucleic acids and/or libraries thereof of small and large nucleic acids are generated simultaneously (i.e., within the same reaction and/or under the same reaction conditions). In some instances, a reaction of the present methods for simultaneously generating strand specific nucleic acids and/or libraries thereof of small and large nucleic acids may not require any additional steps as compared to a corresponding method for generating strand specific nucleic acids for only small or large target nucleic acids. A non-limiting example of small and large target nucleic acids for which strand specific nucleic acids and/or libraries thereof may be simultaneously generated include miRNAs and mRNAs.

Useful target nucleic acids include DNAs, including single stranded DNA (ssDNA) and double stranded DNA (dsDNA). DNA target nucleic acids may be derived from a variety of sources including e.g., plasmids or other source of DNA (including e.g., synthetically derived DNA, DNA obtained from an bacterial genome, DNA obtained from an archaea genome, DNA obtained from an eukaryotic genome, DNA obtained from a bacteriophage, DNA obtained from a virus, etc.). Target DNA may be unmodified or modified, including e.g., using various cloning methods including e.g., restriction digestion, PCR, rolling circle replication, etc. DNA segments of any desired sequence and of essentially any reasonable length may be custom ordered, e.g., as synthesized DNA “blocks” e.g., as available as “gBlocks” from Integrated DNA Technologies, Inc. (Coralville, Iowa).

Useful target nucleic acids include RNAs, including single stranded RNA (ssRNA) and double stranded RNA (dsRNA). According to certain embodiments, the target nucleic acids are target ribonucleic acids (target RNA). Target RNAs may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (IncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, or any combination of RNA types thereof or subtypes thereof.

Sources and/or methods of generating target nucleic acids will vary. Target nucleic acids may be present in a target nucleic acid composition (e.g., a defined composition) or a biological sample (e.g., a sample obtained from or containing a living organism and/or living cells). Biological samples containing target nucleic acids may be prepared, by any convenient means, to render the nucleic acids of the sample available to components of the herein described methods (e.g., primers, oligonucleotides, etc.).

Methods of preparing biological samples containing target nucleic acids will vary. Useful processes may include but are not limited to e.g., homogenizing the sample, lysing one or more cell types of the sample, enriching the same for desired nucleic acids, removing one or more components present in the sample (e.g., proteins, lipids, contaminating nucleic acids), performing nucleic acid isolation to isolate the target nucleic acids, etc. In some instances, cells of a biological sample may be prepared by lysing the cells of the sample. Useful processes for lysing cells include but are not limited to e.g., chemical lysis, enzymatic lysis, mechanical lysis, freeze/thaw lysis, and the like. In some instances, the cells of the sample may not be fixed prior use of target nucleic acid obtained from the cells or a cell of the sample in a method as described herein. In some instances, the cells of the sample may be fixed prior use of target nucleic acid obtained from the cells or a cell of the sample in a method as described herein.

The number of distinct target nucleic acids of differing sequence in a given target nucleic acid composition may vary. While the number of distinct target nucleic acids in a given target nucleic acid composition may vary, in some instances the number of distinct target nucleic acids in a given target nucleic acid composition ranges from 1 to 10⁸, such as 1 to 10⁷, including 1 to 10⁵.

The target nucleic acid composition employed in such methods may be any suitable nucleic acid sample. The nucleic acid sample that includes the target nucleic acid may be combined into the reaction mixture in an amount sufficient for producing the product nucleic acid. According to one embodiment, the nucleic acid sample is combined into the reaction mixture such that the final concentration of nucleic acid in the reaction mixture is from 1 μg/μL to 10 μg/μL, such as from 1 μg/μL to 5 μg/μL, such as from 0.001 μg/μL to 2.5 μg/μL, such as from 0.005 μg/μL to 1 μg/μL, such as from 0.01 μg/μL to 0.5 μg/μL, including from 0.1 μg/μL to 0.25 μg/μL. In certain aspects, the nucleic acid sample that includes the target nucleic acid is isolated from a single cell, e.g., as described in greater detail below. In other aspects, the nucleic acid sample that includes the target nucleic acid is isolated from 2, 3, 4, 5, 6, 7, 8, 9, 10 or more, 20 or more, 50 or more, 100 or more, or 500 or more cells. According to certain embodiments, the nucleic acid sample that includes the target nucleic acid is isolated from 500 or less, 100 or less, 50 or less, 20 or less, 10 or less, 9, 8, 7, 6, 5, 4, 3, or 2 cells.

The target nucleic acid may be present in any nucleic acid sample of interest, including but not limited to, a nucleic acid sample isolated from a single cell, a plurality of cells (e.g., cultured cells), a tissue, an organ, or an organism (e.g., mouse, rat, or the like). In certain aspects, the nucleic acid sample is isolated from a cell(s), tissue, organ, and/or the like of a mammal (e.g., a human, a rodent (e.g., a mouse), or any other mammal of interest). In other aspects, the nucleic acid sample is isolated from a source other than a mammal, such as amphibians (e.g., frogs (e.g., Xenopus)), fish (zebrafish (Danio rerio), or any other non-mammalian nucleic acid sample source.

Approaches, reagents and kits for isolating nucleic acids from such sources are known in the art. For example, kits for isolating nucleic acids from a source of interest—such as the NucleoSpin®, NucleoMag® and NucleoBond® genomic DNA or RNA isolation kits by Takara Bio USA, Inc. (Mountain View, Calif.)—are commercially available. In certain aspects, the nucleic acid is isolated from a fixed biological sample, e.g., formalin-fixed, paraffin-embedded (FFPE) tissue. Nucleic acids from FFPE tissue may be isolated using commercially available kits—such as the NucleoSpin® FFPE DNA or RNA isolation kits by Takara Bio USA, Inc. (Mountain View, Calif.).

In some instances, the methods described herein may include denaturing the target nucleic acid, e.g., by subjecting a reaction mixture containing the target nucleic acid to a temperature sufficient to denature secondary structure of the target nucleic acid. Depending on the context, denaturing may take place before or after one or more reaction components have been added to the reaction mixture and, in some instances, is performed prior to the start of transcription, e.g., reverse transcription to generate the single product nucleic acid. Useful denaturing temperatures will vary and may range from less than 50° C. to more than 100° C., including but not limited to e.g., 50° C. or more, 55° C. or more, 65° C. or more, 70° C. or more, 72° C. or more, 75° C. or more, 80° C. or more, 85° C. or more, 90° C. or more, 95° C. or more, etc.

Various processing steps can be applied to the target nucleic acid prior to the coupling of an adaptor, e.g., the first adaptor, thereto. Examples of processing of the target nucleic acid include, but are not limited to, one or more steps involving purification, fragmentation (including chemical, physical, and enzymatic fragmentation and the like), size-selection, RNase treatment, DNase treatment, denaturation, hybridization or annealing, labeling, reverse transcription, and amplification. Embodiments include, but are not limited to, e.g., where the target nucleic acid is genomic DNA, cell-free DNA, cell-free fetal DNA, DNA isolated from formalin fixed paraffin embedded (FFPE) tissue, DNA isolated from fixed cells, single-stranded DNA, RNA, mRNA, cellular RNA depleted for rRNA and enriched for mRNA, RNA isolated from FFPE tissue, RNA isolated from fixed cells, fragmented preparations of each of the preceding target nucleic acids, and combinations of each of the preceding target nucleic acids.

Samples from which target nucleic acids may be obtained or derived will vary. Cellular samples may be derived from a variety of sources including but not limited to e.g., a cellular tissue, a biopsy, a blood sample, a cell culture, etc. Additionally, cellular samples may be derived from specific organs, tissues, tumors, neoplasms, or the like. Furthermore, cells from any population can be the source of a cellular sample used in the subject methods, such as a population of prokaryotic or eukaryotic single celled organisms including bacteria or yeast.

As such, in some instances, the source of an RNA sample utilized in the subject methods may be a mammalian cellular sample, such as a rodent (e.g., mouse or rat) cellular sample, a non-human primate cellular sample, a human cellular sample, or the like. In some instances, a mammalian cellular sample may be mammalian blood sample, including but not limited to e.g., a rodent (e.g., mouse or rat) blood sample, a non-human primate blood sample, a human blood sample, or the like.

As summarized above, in some instances, a nucleic acid sample may be derived from a single cell to generate a one or more libraries as described herein. Such “single cell libraries” may then be employed in further downstream applications, such as sequencing applications. As used herein, a “single cell” refers to one cell. Single cells useful as the source of template RNAs and/or in generating single cell libraries, such as expression libraries and/or immune cell receptor repertoire libraries can be obtained from a tissue of interest, or from a biopsy, blood sample, or cell culture. Additionally, cells from specific organs, tissues, tumors, neoplasms, or the like can be obtained and used in the methods described herein.

Single cells, for use in such methods, may be obtained by any convenient method. For example, in some instances, single cells may be obtained through limiting dilution of cellular sample. In some instances, the present methods may include a step of obtaining single cells. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example a 96-well plate, 384 well plate, or a plate with any number of wells such as 2000, 4000, 6000, or 10000 or more. The multi-well plate can be part of a chip and/or device. The present disclosure is not limited by the number of wells in the multi-well plate. In various embodiments, the total number of wells on the plate is from 100 to 200,000, or from 5000 to 10,000. In other embodiments the plate comprises smaller chips, each of which includes 5,000 to 20,000 wells. For example, a square chip may include 72 by 72 nanowells, with a diameter of 0.01-0.5 mm.

In some instances, single cells may be obtained by sorting a cellular sample using a cell sorter instrument. By “cell sorter” as used herein is meant any instrument that allows for the sorting of individual cells into an appropriate vessel for downstream processes, such as those processes of library preparation as described herein.

Useful cell sorters include flow cytometers, such as those instruments utilized in fluorescence activated cell sorting (FACS). Flow cytometry is a well-known methodology using multi-parameter data for identifying and distinguishing between different particle (e.g., cell) types i.e., particles that vary from one another terms of label (wavelength, intensity), size, etc., in a fluid medium. In flow cytometrically analyzing a sample, an aliquot of the sample is first introduced into the flow path of the flow cytometer. When in the flow path, the cells in the sample are passed substantially one at a time through one or more sensing regions, where each of the cells is exposed separately individually to a source of light at a single wavelength (or in some instances two or more distinct sources of light) and measurements of scatter and/or fluorescent parameters, as desired, are separately recorded for each cell. The data recorded for each cell is analyzed in real time or stored in a data storage and analysis means, such as a computer, for later analysis, as desired.

Cells sorted using a flow cytometer may be sorted into a common vessel (i.e., a single tube), or may be separately sorted into individual vessels. For example, in some instances, cells may be sorted into individual wells of a multi-well plate, as described below.

Useful cell sorters also include multi-well-based systems that do not employ flow cytometry. Such multi-well based systems include essentially any system where cells may be deposited into individual wells of a multi-well container by any convenient means, including e.g., through the use of Poisson distribution (i.e., limiting dilution) statistics (e.g., multi-sample nanodispense (MSND) systems), individual placement of cells (e.g., through manual cell picking or dispensing using a robotic arm or pipettor). In some instances, useful multi-well systems include a multi-well wafer or chip, where cells are deposited into the wells or the wafer/chip and individually identified by a microscopic analysis system. In some instances, an automated microscopic analysis system may be employed in conjunction with a multi-well wafer/chip to automatically identify individual cells to be subjected to downstream analyses, including library preparation, as described herein.

In some instances, one or more cells may be sorted into or otherwise transferred to an appropriate reaction vessel, where such vessels include those sufficient for performing one or more of the aspects of library preparation as described herein. Reaction components may be added to reaction vessels, including e.g., components for preparing an RNA sample, components for generating a product double stranded cDNA, components for one or more library preparation reactions, etc. Reaction vessels into which the reaction mixtures and components thereof may be added and within which the reactions of the subject methods may take place will vary. Useful reaction vessels include but are not limited to e.g., tubes (e.g., single tubes, multi-tube strips, etc.), wells (e.g., of a multi-well plate (e.g., a 96-well plate, 384 well plate, or a plate with any number of wells such as 2000, 4000, 6000, or 10000 or more). Multi-well plates may be independent or may be part of a chip and/or device.

In some instances, reaction mixtures and components thereof may be added to and the reactions of the subject methods may take place in a liquid droplet (e.g., a water-oil emulsion droplet), e.g., as described in more detail below. Whereas the droplets may serve the purpose of individual reaction vessels, the droplets (or emulsion containing droplets) will generally be housed in a suitable container such as, e.g., a tube or well or microfluidic channel. Amplification reactions performed in droplets may be sorted, e.g., based on fluorescence (e.g., from nucleic acid detection reagent or labeled probe), using a fluorescence based droplet sorter. Useful fluorescence based droplet sorters will vary and may include e.g., a flow cytometers, microfluidic-based droplet sorters, and the like.

As indicated above, in protocols that include a pooling step, the pooling step can be performed after production of a product double stranded cDNA, e.g., from a single cell, from a droplet, from a well, etc. As such, in certain embodiments of the methods described herein, cells are obtained from a tissue of interest (e.g., blood) and a single-cell suspension is obtained. A single cell is placed in one well of a multi-well plate, or other suitable container, such as a microfluidic chamber or tube. The cells are lysed and reaction components are added directly to the lysates. Whether or not pooling of single cells samples is employed the generated libraries may be sequenced to produce reads. This may allow identification of genes that are expressed in each single cell.

In certain embodiments of the methods described herein, droplets are obtained and a single droplet is sorted into one well of a multi-well plate, or other suitable container, such as a microfluidic chamber or tube. The reaction mixture may be added directly to the droplet, e.g., without additional purification.

In some instances, the methods may include the step of obtaining single droplets. Obtaining droplets cells may be done according to any convenient protocol, including e.g., mechanically sorting droplets (e.g., utilizing a fluorescence based sorter (e.g., a flow cytometer or microfluidic-based sorter). Single droplets can be placed in any suitable reaction vessel in which single droplets can be treated individually. For example a 96-well plate, 384 well plate, or a plate with any number of wells such as 2000, 4000, 6000, or 10000 or more. The multi-well plate can be part of a chip and/or device. The present disclosure is not limited by the number of wells in the multi-well plate. In various embodiments, the total number of wells on the plate is from 100 to 200,000, or from 5000 to 10,000. In other embodiments the plate comprises smaller chips, each of which includes 5,000 to 20,000 wells. For example, a square chip may include 72 by 72 or 125 by 125 nanowells. The nanowells can comprise a diameter of 0.01-0.5 mm. The wells (e.g., nanowells) in the multi-well plates may be fabricated in any convenient size, shape or volume. The well may be 100 μm to 1 mm in length, 100 μm to 1 mm in width, and 100 μm to 1 mm in depth. In various embodiments, each nanowell has an aspect ratio (ratio of depth to width) of from 1 to 4. In one embodiment, each nanowell has an aspect ratio of 2. The transverse sectional area may be circular, elliptical, oval, conical, rectangular, triangular, polyhedral, or in any other shape. The transverse area at any given depth of the well may also vary in size and shape.

In certain embodiments, the wells have a volume of from 0.1 nl to 1 μl. The nanowell may have a volume of 1 μl or less, such as 500 nl or less. The volume may be 200 nl or less, such as 100 nl or less. In an embodiment, the volume of the nanowell is 100 nl. Where desired, the nanowell can be fabricated to increase the surface area to volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments.

The wells can be designed such that a single well includes a single cell or a single droplet. An individual cell or droplet may also be isolated in any other suitable container, e.g., microfluidic chamber, droplet, nanowell, tube, etc. Any convenient method for manipulating single cells or droplets may be employed, where such methods include fluorescence activated cell sorting (FACS), robotic device injection, gravity flow, or micromanipulation and the use of semi-automated cell pickers (e.g. the Quixell™ cell transfer system from Stoelting Co.), etc. In some instances, single cells or droplets can be deposited in wells of a plate according to Poisson statistics (e.g., such that approximately 10%, 20%, 30% or 40% or more of the wells contain a single cell or droplet—which number can be defined by adjusting the number of cells or droplets in a given unit volume of fluid that is to be dispensed into the containers). In some instances, a suitable reaction vessel comprises a droplet (e.g., a microdroplet). Individual cells or droplets can, for example, be individually selected based on features detectable by microscopic observation, such as location, morphology, the presence of a reporter gene (e.g., expression), the presence of a bound antibody (e.g., antibody labelling), FISH, the presence of an RNA (e.g., intracellular RNA labelling), or qPCR.

Following obtainment of a desired cell population or single cells, e.g., as described above, nucleic acids can be released from the cells by lysing the cells. Lysis can be achieved by, for example, heating or freeze-thaw of the cells, or by the use of detergents or other chemical methods, or by a combination of these. However, any suitable lysis method can be used. In some instances, a mild lysis procedure can advantageously be used to prevent the release of nuclear chromatin, thereby avoiding genomic contamination of a cDNA library, and to minimize degradation of mRNA. For example, heating the cells at 72° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Publication No. 2007/0281313).

Where desired, a given single cell or droplet workflow may include a pooling step where a nucleic acid product composition, e.g., made up of product double stranded cDNA, is combined or pooled with the nucleic acid product compositions obtained from one or more additional cells or droplets. The number of different nucleic acid product compositions produced from different cells or droplets that are combined or pooled in such embodiments may vary, where the number ranges in some instances from 2 to 50, such as 3 to 25, including 4 to 20 to 1,000 to 1,700, to 2000 or 10,000, or more.

In some embodiments, a multi-sample nano-dispenser (MSND) system that includes a multiwell plate, e.g., in the form of an array of addressable nanowells, and a sample dispener is employed. An example of such a MSND system is the ICELL8® Single-Cell MSND System (Wafergen, Fremont, Ca). Details of the ICELL8® MSND system are further found in U.S. Pat. Nos. 7,833,709 and 8,252,581, as well as published United States Patent Application Publication Nos. 2015/0362420 and 2016/0245813, the disclosures of which are herein incorporated by reference.

In some instances, the method is performed in the same container, where in some instances the container is selected from the group consisting of: a microtiter plate, a droplet, a microfluidic device, or any combination thereof. In some instances, the container includes a fluidically isolated microwell in a microwell array.

### Primers

As summarized above, the methods of the present disclosure may employ primers. Primers may be employed at various points within the herein described methods including but not limited to e.g., where a primer is contacted with a target nucleic acid and employed in a primer extension reaction to generate the complement of the target nucleic acid. Primers may also be employed amplification reactions and the like.

Useful primers can include a blocked 5′-end and an extendible 3′-end. The blocked 5′-end can include one or more blocking groups that prevent modification of the respective terminus by one or more enzyme activities or chemical modifications. For example, the blocking group can prevent ligation at the blocked terminus, polymerase extension at the blocked terminus, or exonuclease activity at the blocked terminus. The extendible 3′-end is extendible by a polymerase and can include a 3′-hydroxyl group. The primer can include a barcode.

A primer may be employed in an elongation reaction to generate a first strand product nucleic acid, also referred to herein as a single product nucleic acid. Such a primer may also be referred to as a single product nucleic acid primer or a single product nucleic acid synthesis primer (e.g., a first strand cDNA primer) or a first strand primer. Such primers may include a domain that hybridizes to at least a portion of the adaptor, including where the adaptor is ligated to the target nucleic acid. First strand primers of the presently described methods may or may not hybridize, in whole or in part, to the target nucleic. First strand primers may or may not include one or more additional domains which may be viewed as a second (e.g., 5′) domain that does not hybridize to the template nucleic acid, e.g., a non-template sequence domain as described in more detail below. While the length of the adaptor binding domain may vary, in some instances the length of this domain ranges from 5 to 50 nts, such as 6 to 25 nts, e.g., 6 to 20 nts.

The primers may or may not include one or more nucleotides (or analogs thereof) that are modified or otherwise non-naturally occurring. For example, a primer may include one or more nucleotide analogs (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the primer.

In some instances, a primer may include a 5′ adapter sequence (e.g., a defined nucleotide sequence 5′ of the 3′ hybridization domain of the primer), the 5′ adapter sequence may serve various purposes in downstream applications. In some instances, the 5′ adapter sequence may serve as a primer binding site for further amplification or, e.g., nested amplification or suppression amplification.

In some instances, one or more of the primers or oligonucleotides employed (including e.g., single product nucleic acid primers, template switch oligonucleotides, etc.) may include two or more domains. For example, the primer or oligonucleotide may include a first (e.g., 3′) domain that hybridizes to a template or an adaptor and a second (e.g., 5′) domain that does not hybridize to a template or an adaptor. In some instances, the sequence of the first and second domains may be independently defined or arbitrary.

The primer can include an affinity tag that can be used to isolate the adaptor-coupled target nucleic acid. Isolation of the adaptor-coupled target nucleic acid using the affinity tag can include isolating the adaptor-coupled target nucleic acid from any uncoupled first adaptor and any uncoupled target nucleic acid. Extension of the primer to form the first primer extension product complementary to the portion of the adaptor-coupled target nucleic acid can be performed using a polymerase. For example, where the target nucleic acid includes RNA, extension of the primer can include reverse transcription using an RNA-dependent DNA polymerase, and where the target nucleic acid includes DNA, a complementary nucleic acid strand can be synthesized by primer extension using a DNA-dependent DNA polymerase. Useful RNA-dependent DNA polymerases include reverse transcriptases, such as retroviral reverse transcriptase and recombinant or modified forms thereof. Useful reverse transcriptases include murine leukemia virus reverse transcriptases and recombinant or modified forms thereof. Useful DNA-dependent DNA polymerases include various prokaryotic or eukaryotic DNA polymerases and recombinant or modified forms thereof.

Addition of the at least one non-template directed nucleotide to the 3′-end of the first primer extension product can be performed using terminal transferase, including an RNA-dependent DNA polymerase having terminal transferase activity. One example of an RNA-dependent DNA polymerase having terminal transferase activity is murine leukemia virus reverse transcriptase. Terminal transferase activity can add a plurality of non-template directed nucleotides, including from one to six or more non-template directed nucleotides. In some embodiments, the terminal transferase activity can add about three or more non-template directed nucleotides. Non-template directed nucleotides added to the 3′-end of the first primer extension product can result in a homopolymeric stretch of nucleotides at the 3′-end. In certain embodiments, the at least one non-template directed nucleotide includes a homopolymer of cytosines at the 3′-end of the first primer extension product.

### Template Switch Oligonucleotide

A template-switching reaction, such as but not limited to template-switching reverse transcription reactions, may make use of a template switch oligonucleotide. By “template switch oligonucleotide” is meant an oligonucleotide template to which a polymerase switches from an initial template (e.g., template nucleic acid (e.g., a DNA or RNA template)) during a nucleic acid polymerization reaction. In this regard, the template may be referred to as a “donor template” and the template switch oligonucleotide may be referred to as an “acceptor template.” As used herein, an “oligonucleotide” is a single-stranded multimer of nucleotides from 2 to 500 nucleotides, e.g., 2 to 200 nucleotides. Oligonucleotides may be synthetic or may be made enzymatically, and, in some embodiments, are 10 to 50 nucleotides in length. Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 10 to 20, 21 to 30, 31 to 40, 41 to 50, 51-60, 61 to 70, 71 to 80, 80 to 100, 100 to 150 or 150 to 200, up to 500 or more nucleotides in length, for example.

The second adaptor can be referred to as a template switch oligonucleotide (e.g., the terms “second adaptor” and “template switch oligonucleotide” can be used interchangeably). As used herein “template switch oligonucleotide” or “second adaptor” can generally refer to an oligonucleotide template to which a polymerase switches from an initial template (e.g., a template RNA, DNA, or cDNA) during a nucleic acid polymerization reaction. In this regard, a target nucleic acid may be referred to as a “donor template” and the template switch oligonucleotide may be referred to as an “acceptor template.” As used herein, an “oligonucleotide” can refer to a single-stranded multimer of nucleotides from 2 to 500 bases, e.g., 2 to 200 bases. Oligonucleotides may be synthetic or may be made enzymatically, and, in some embodiments, are 10 to 50 bases in length. Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 10 to 20, 21 to 30, 31 to 40, 41 to 50, 51 to 60, 61 to 70, 71 to 80, 80 to 100, 100 to 150 or 150 to 200, up to 500 or more bases in length, for example.

In some instances, the template switch oligonucleotide includes a domain that specifically binds to a surface-attached sequencing platform oligonucleotide, a sequencing primer binding domain, a barcode domain, a barcode sequencing primer binding domain, a molecular identification domain, and combinations thereof. In some instances, the template switch oligonucleotide comprises a modification that prevents the polymerase from switching from the template switch oligonucleotide to a different template nucleic acid after synthesizing the complement of the 5′ adapter sequence. In some instances, the modification is selected from the group consisting of: an abasic lesion, a nucleotide adduct, an iso-nucleotide base, and combinations thereof. In some instances, the template switch oligonucleotide comprises one or more nucleotide analogs.

As summarized above, the template switch oligonucleotide may comprise one or more modified nucleotides, nucleotide analogs, or non-naturally occurring nucleotides. The template switch oligonucleotide may include one or more nucleotide analogs such as LNA, FANA, 2′-O-Me RNA, 2′-fluoro RNA, or others. Other modifications may be included, such as linkage modifications, 5′ or 3′ end modifications, or other modifications which provide a desired functionality. The template switch oligonucleotide can include a modification that prevents the polymerase from switching from the template switch oligonucleotide to a different template nucleic acid after synthesizing the compliment of the 5′ end of the template switch oligonucleotide (e.g., a 5′ adapter sequence of the template switch oligonucleotide). Useful modifications include, but are not limited to, an abasic lesion (e.g., a tetrahydrofuran derivative), a nucleotide adduct, an iso-nucleotide base (e.g., isocytosine, isoguanine, and/or the like), and any combination thereof.

In certain aspects, the template switch oligonucleotide includes a 3′ hybridization domain. The 3′ hybridization domain may vary in length, and in some instances ranges from 2 to 10 nts in length, such as 3 to 7 nts in length. The 3′ hybridization domain of a template switch oligonucleotide may include a sequence complementary to a non-templated sequence added to a single product nucleic acid of the template-switching reaction (e.g., a cDNA). Non-templated sequences generally refer to those sequences that do not correspond to and are not templated by a template, e.g., a RNA template or a DNA template. Where present in the 3′ hybridization domain of a template switch oligonucleotide, non-templated sequences may encompass the entire 3′ hybridization domain or a portion thereof. In some instances, a non-templated sequence may include or consist of a hetero-polynucleotide, where such a hetero-polynucleotide may vary in length from 2 to 10 nts in length, such as 3 to 7 nts in length, including 3 nts. In some instances, a non-templated sequence may include or consist of a homo-polynucleotide, where such a homo-polynucleotide may vary in length from 2 to 10 nts in length, such as 3 to 7 nts in length, including 3 nts.

In some instances, the template switch oligonucleotide (i.e., second adaptor) can include a blocked 5′-end and can include a blocked 3′-end. The second adaptor can also include a barcode. The blocked 3′-end can be complementary to the at least one non-template directed nucleotide at the 3′-end of the primer extension product. For example, the blocked 3′-end of the second adaptor can include homopolymeric stretch of nucleotides that are complementary to a homopolymeric stretch of nucleotides at the 3′-end of the primer extension product. In certain embodiments, the blocked 3′-end of the second adaptor can include a homopolymeric stretch of guanosines that are complementary to a homopolymer of cytosines at the 3′-end of the first primer extension product, where the homopolymer of cytosines forms the at least one non-template directed nucleotide of the primer extension product. In this manner, base pairing between the respective homopolymers can permit template strand switching by a polymerase, such as a reverse transcriptase, to further extend the primer extension product using the second adaptor as a template and form the second primer extension product. Extending the at least one non-template directed nucleotide at the 3′-end of the primer extension product to form the second primer extension product can be performed using an RNA-dependent DNA polymerase capable of template switching, such as murine leukemia virus reverse transcriptase.

The terms “first primer extension product” and “second primer extension product”, as used herein with respect to template switching reactions, may refer to different intermediates of a primer extension reaction. Put another way, a single extension product, extended from a single primer along two templates via a template switching reaction, may generate a “first primer extension product” that is an intermediate of a “second primer extension product”, where the first and second primer extension products represent the same polynucleotide at different points during the primer extension reaction. Generally, but not necessarily, a first primer extension product will refer to an intermediate polynucleotide comprising sequence complementary to a first template of a template switching reaction and a second primer extension product will refer to a polynucleotide comprising sequence complementary to the first template and a second template of the template switching reaction. The second primer extension product may, in some instances, also refer to a product nucleic acid of a template switching reaction, such as e.g., a single product nucleic acid polymerized in a template switching reaction.

The second primer extension product can therefore include, in a 5′ to 3′ direction, the primer, a complement of the target nucleic acid, at least one non-template directed nucleotide (e.g., a homocysteine polymeric stretch), and a complement to the second adaptor.

A template switch oligonucleotide can be free in solution or can be attached to a solid support (e.g., a bead). In some instances, a template switch oligonucleotide is dried in a container (e.g., a multi well array chip). The dried template switch oligonucleotide can be covalently or non-covalently attached to the container.

### Amplification and Sequencing

The second primer extension product (i.e., single product nucleic acid) can be amplified in various ways and/or sequenced in various ways. For example, the second primer extension product can be amplified by performing a polymerase chain reaction using a first amplification primer including a portion of a sequence of the second adaptor and a second amplification primer including a portion of a sequence of the primer or a portion of a sequence complementary to a portion of the first adaptor. At least one of the first amplification primer and the second amplification primer can include a barcode. The barcode can include a unique sequence that can serve to identify a particular amplification product. Barcodes can include defined sequences or can include randomized sequences. The first amplification primer and the second amplification primer can include different barcodes. In this manner, the origin of a particular amplification product can be identified and tracked following sequencing of the amplification product. Identification and tracking can further facilitate multiplexing applications using such amplified products, including amplified products from one or more libraries derived from one or more sources of target nucleic acid. The first amplification primer and the second amplification primer can also include standard sequences for use on various amplification and/or sequencing platforms; e.g., P5 and P7 sequences that bind the flow cell used in the Illumina Genome Analyzer System. The first amplification primer and second amplification primer can also include sequences for use in further amplification reactions (e.g., primer binding site, polymerase binding site). The first amplification primer and the second amplification primer can also include any non-templated sequence (e.g., a sequence that is not complementary to that in a target nucleic acid).

Amplification may be performed in a single round or multiple rounds of amplification may be employed. For example, in some instances, after a first round of amplification one or more amplification primers not utilized in the first round may be added to the reaction mixture to facilitate a second round of amplification using the product of the first round of amplification as a nucleic acid template. In some instances, the second or subsequent round(s) of amplification may involve nested amplification, i.e., where the primer binding sites utilized in the second or subsequent round(s) of amplification are within (i.e., one or more nucleotides from the 3′ or 5′ end) of the product generated in the first round of amplification. Where employed, the degree of nesting will vary as desired including e.g., where the second or subsequent primer binding site is one or more, including 2 or more, 3 or more, 4 or more, 5 or more, 6 or more, 7 or more, 8 or more, 9 or more, 10 or more, 15 or more, 20 or more, etc., nucleotides from the 3′ or 5′ end of the amplicon generated in the first round of amplification.

In some instances, second or subsequent round(s) amplification will not be nested, including where the second round of amplification makes use of one or more primer binding sites utilized in the prior round of amplification or a primer binding site added during the prior round of amplification (e.g., a primer binding site added as part of a non-templated sequence). In some instances, a second or subsequent round of amplification may make use of a nested primer amplification site at one end and a non-nested (e.g., a prior used primer binding site or an added primer binding site) at the other end, including where the nested site is at the 3′ end of the amplicon or the 5′ end of the amplicon.

Following prescribed library amplification steps, the prepared libraries may be considered ready for sequencing. In certain embodiments, the methods provided may further include subjecting a prepared library to an NGS protocol. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

In some instances, methods provided may include the use of an amplification polymerase, e.g., for use in amplifying a produced double stranded cDNA, a produced nucleic acid library, etc. Any convenient amplification polymerase may be employed including but not limited to DNA polymerases including thermostable polymerases. Useful amplification polymerases include e.g., Taq DNA polymerases, Pfu DNA polymerases, derivatives thereof and the like. In some instances, the amplification polymerase may be a hot start polymerase including but not limited to e.g., a hot start Taq DNA polymerase, a hot start Pfu DNA polymerase, and the like.

An amplification polymerase may be combined into a reaction mixture such that the final concentration of the amplification polymerase is sufficient to produce a desired amount of the product nucleic acid, e.g., a desired amount of amplified product double stranded cDNA, a desired amount of library nucleic acid, etc. In certain aspects, the amplification polymerase (e.g., a thermostable DNA polymerase, a hot start DNA polymerase, etc.) is present in the reaction mixture at a final concentration of from 0.1 to 200 units/μL (U/μL), such as from 0.5 to 100 U/μL, such as from 1 to 50 U/μL, including from 5 to 25 U/μL, e.g., 20 U/μL.

### Non-Templated Sequences

Aspects of the described methods may, in some instances, include the use of non-templated sequences. The terms “non-templated sequence” and “non-template sequence” generally refer to those sequences involved in the subject method that do not correspond to the template (e.g., are not present in the templates, do not have a complementary sequence in the template or are unlikely to be present in or have a complementary sequence in the template). Non-templated sequences are those that are not templated by a template, e.g., a RNA or DNA template, thus they may be, e.g., added during an elongation reaction in the absence of corresponding template, e.g., nucleotides added by a polymerase having non-template directed terminal transferase activity. The addition of non-templated sequence to a nucleic acid need not be necessarily limited to elongation reaction. For example, in some instances, a non-templated sequence may be added through ligation of the non-templated sequence to the nucleic acid.

Non-template and non-templated sequence may, but not exclusively, refer to those sequences present on a primer, template switch oligonucleotide, etc., that do not hybridize to the nucleic acid template (such sequences may, in some instances, be referred to as non-hybridizing sequence). Non-templated sequence will vary, in both size and composition. In some instances, non-templated sequence, e.g., non-templated sequence present on a template switch oligonucleotide or a primer, may range from 10 nt to 1000 nt or more including but not limited to e.g., 10 nt to 900 nt, 10 nt to 800 nt, 10 nt to 700 nt, 10 nt to 600 nt, 10 nt to 500 nt, 10 nt to 400 nt, 10 nt to 300 nt, 10 nt to 200 nt, 10 nt to 100 nt, 10 nt to 90 nt, 10 nt to 80 nt, 10 nt to 70 nt, 10 nt to 60 nt, 10 nt to 50 nt, 10 nt to 40 nt, 10 nt to 30 nt, 10 nt to 20 nt, etc.

In some instances, a non-templated sequence, as noted above, may be included in the 3′ hybridization domain of a template switch oligonucleotide. When present in the 3′ hybridization domain of a template switch oligonucleotide, a non-templated sequence may include or consist of a hetero-polynucleotide, where such a hetero-polynucleotide may vary in length from 2 to 10 nts in length, such as 3 to 7 nts in length, including 3 nts. In some instances, a non-templated sequence present in the 3′ hybridization domain of a template switch oligonucleotide may include or consist of a homo-polynucleotide, where such a homo-polynucleotide may vary in length from 2 to 10 nts in length, such as 3 to 7 nts in length, including 3 nts.

Non-templated sequences present on an oligonucleotide or a primer may be present at the 5′ end of the oligonucleotide or primer and may, in such instances, be referred to as a 5′ non-templated sequence. In some instances, only one oligonucleotide or primer may include a non-templated sequence (e.g., a 5′ non-templated sequence) in a subject reaction. In some instances, two or more oligonucleotides and/or primers utilized in a subject reaction may include a non-templated sequence (e.g., a 5′ non-templated sequence). Where two or more oligonucleotides and/or primers include a non-templated sequence, different non-templated sequences may be employed. In some instances, where two or more oligonucleotides and/or primers have a 5′ non-templated sequence, such sequences may have the same 5′ non-templated sequence.

In some instances, non-templated sequence, including e.g., 5′ non-templated sequence, may include one or more restriction endonuclease recognition sites. In some instances, one or more restriction endonuclease recognition sites may be incorporated into a subject nucleic acid allowing manipulation of the produced nucleic acid, e.g., by cleaving the subject nucleic acid at one or more of the incorporated restriction endonuclease recognition sites.

In some instances, non-templated sequence, including e.g., 5′ non-templated sequence, may include one or more primer binding sites. In some instances, one or more primer binding sites may be incorporated into a subject nucleic acid allowing further amplification of the produced nucleic acid, including e.g., amplifying all or a portion of the nucleic acid using one or more of the primer binding sites.

Useful primer binding sites will vary widely depending on the desired complexity of the primer binding site and the corresponding primer. In some instances, useful primer binding sites include those having complementarity to a ∥ A primer (e.g., as available from Takara Bio USA, Inc., Mountain View, Calif.). According to one embodiment, an oligonucleotide or a primer utilized in generating a product double stranded cDNA includes a non-template sequence that includes a ∥ A primer binding site. According to one embodiment, a nucleic acid utilized in an end capturing reaction includes a non-template sequence that includes a ∥ A primer binding site.

In some instances, non-templated sequence, including e.g., 5′ non-templated sequence, may include one or more barcode sequences, In some instances, such barcode sequences may be or may include a unique molecular identifier (UMI) domain. UMI nucleic acids, and their use in various applications, are further described in published United States Patent Application Publication No. US20150072344; the disclosure of which is incorporated herein by reference in their entirety.

In some instances, one or more barcode sequences of a non-templated sequence may provide for retrospective identification of the source of a generated nucleic acid, e.g., following a sequencing reaction where the barcode is sequenced. For example, in some instances, a non-templated sequence that includes a barcode specific for the source (e.g., sample, well, cell, etc.) of the template is incorporated during a reaction. Such source identifying barcodes may be referred to herein as a “source barcode sequence” and such sequences may vary and may be assigned a term based on the source that is identified by the barcode. Source barcodes may include e.g., a sample barcode sequence that retrospectively identifies the sample from which the sequenced nucleic acid was generated, a well barcode sequence that retrospectively identifies the well (e.g., of a multi-well plate) from which the sequenced nucleic acid was generated, a droplet barcode sequence that retrospectively identifies the droplet from which the sequenced nucleic acid was generated, a cell barcode sequence that retrospectively identifies the cell (e.g., of a multi-cellular sample) from which the sequenced nucleic acid was generated, etc. Barcodes may find use in various procedures including e.g., where nucleic acids are pooled following barcoding, e.g., prior to sequencing.

In some instances, a non-templated sequence, e.g., present on an oligonucleotide and/or a nucleic acid primer, includes a sequencing platform adapter construct. By “sequencing platform adapter construct” is meant a nucleic acid construct that includes at least a portion of a nucleic acid domain (e.g., a sequencing platform adapter nucleic acid sequence) or complement thereof utilized by a sequencing platform of interest, such as a sequencing platform provided by Illumina® (e.g., the HiSeq™, MiSeg™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Oxford Nanopore™ (e.g., MinION sequencing system), Life Technologies™ (e.g., a SOLiD sequencing system); Roche (e.g., the 454 GS FLX+ and/or GS Junior sequencing systems); or any other sequencing platform of interest.

In certain aspects, a non-templated sequence includes a sequencing platform adapter construct that includes a nucleic acid domain that is a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind). The sequencing platform adapter constructs may include nucleic acid domains (e.g., “sequencing adapters”) of any length and sequence suitable for the sequencing platform of interest. In certain aspects, the nucleic acid domains are from 4 to 200 nts in length. For example, the nucleic acid domains may be from 4 to 100 nts in length, such as from 6 to 75, from 8 to 50, or from 10 to 40 nts in length. According to certain embodiments, the sequencing platform adapter construct includes a nucleic acid domain that is from 2 to 8 nts in length, such as from 9 to 15, from 16-22, from 23-29, or from 30-36 nts in length.

The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5, P7, Read 1 primer and Read 2 primer domains employed on the Illumina®-based sequencing platforms. Other example nucleic acid domains include the A adapter and P1 adapter domains employed on the Ion Torrent™-based sequencing platforms.

The nucleotide sequences of non-templated sequence domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of the sequencing platform adapter construct of the non-templated sequence (e.g., a template switch oligonucleotide and/or a single product nucleic acid primer, and/or the like) may be designed to include all or a portion of one or more nucleic acid domains in a configuration that enables sequencing the nucleic acid insert (corresponding to the template nucleic acid) on the platform of interest. Sequencing platform adaptor constructs that may be included in a non-templated sequence as well as other nucleic acid reagents described herein, are further described in U.S. patent application Ser. No. 14/478,978 published as US 2015-0111789 A1, the disclosure of which is herein incorporated by reference.

Non-templated sequence may be added to a nucleic acid of interest, e.g., to an oligonucleotide, a nucleic acid primer, a generated dsDNA, etc., by a variety of means. For example, as noted above, non-templated sequence may be added through the action of a polymerase with terminal transferase activity. Non-templated sequence, e.g., present on a primer or oligonucleotide, may be incorporated into a product nucleic acid during an amplification reaction. In some instances, non-templated nucleic acid sequence may be directly attached to a nucleic acid, e.g., to a primer or oligonucleotide prior to amplification, to a product of nucleic acid amplification, etc. Methods of directly attaching a non-templated sequence to a nucleic acid will vary and may include but are not limited to e.g., ligation, chemical synthesis/linking, enzymatic nucleotide addition (e.g., by a polymerase with terminal transferase activity), and the like.

In some instances, the methods may include attaching sequencing platform adapter constructs to ends of a nucleic acid. For example, in some instances, oligonucleotides and/or primers utilized in the subject methods may not include sequencing platform adapter constructs and thus desired sequencing platform adapter constructs may be attached following the production of a nucleic acid of interest. Adapter constructs attached to the ends of a nucleic acid of interest or a derivative thereof may include any sequence elements useful in a downstream sequencing application, including any of the elements described above with respect to the optional sequencing platform adapter constructs of the oligonucleotides and/or primers of the herein described methods. For example, the adapter constructs attached to the ends of nucleic acid of interest or a derivative thereof may include a nucleic acid domain or complement thereof selected from the group consisting of: a domain that specifically binds to a surface-attached sequencing platform oligonucleotide, a sequencing primer binding domain, a barcode domain, a barcode sequencing primer binding domain, a molecular identification domain, and combinations thereof.

Attachment of the sequencing platform adapter constructs may be achieved using any suitable approach. In certain aspects the adapter constructs are attached to the ends of the product nucleic acid or a derivative thereof using an approach that is the same or similar to “seamless” cloning strategies. Seamless strategies eliminate one or more rounds of restriction enzyme analysis and digestion, DNA end-repair, de-phosphorylation, ligation, enzyme inactivation and clean-up, and the corresponding loss of nucleic acid material. Seamless attachment strategies of interest include: the In-Fusion® cloning systems available from Takara Bio USA, Inc. (Mountain View, Calif.), SLIC (sequence and ligase independent cloning) as described in Li & Elledge (2007) Nature Methods 4:251-256; Gibson assembly as described in Gibson et al. (2009) Nature Methods 6:343-345; CPEC (circular polymerase extension cloning) as described in Quan & Tian (2009) PLoS ONE 4(7): e6441; SLiCE (seamless ligation cloning extract) as described in Zhang et al. (2012) Nucleic Acids Research 40(8): e55, and the GeneArt® seamless cloning technology by Life Technologies (Carlsbad, Calif.).

Any suitable approach may be employed for providing additional nucleic acid sequencing domains to a nucleic acid of interest or derivative thereof having less than all of the useful or necessary sequencing domains for a sequencing platform of interest. For example, the a nucleic acid of interest or derivative thereof could be amplified using PCR primers having adapter sequences at their 5′ ends (e.g., 5′ of the region of the primers complementary to the nucleic acid of interest or derivative thereof), such that the amplicons include the adapter sequences in the original nucleic acid as well as the adapter sequences in the primers, in any desired configuration. Other approaches, including those based on seamless cloning strategies, restriction digestion/ligation, or the like may be employed.

### Libraries

Various strand specific nucleic acid libraries can be formed by the methods provided herein. Where the target nucleic acid is mRNA, for example, the resulting nucleic acid library can prove valuable in analysis of the transcriptome presented by the organism, tissue, or single cell from which the target nucleic acid was derived. By properly ascertaining expressed nucleic acid sequences, it can be possible to annotate the structures of transcribed genes. Such structural information can include the 5′ and 3′ ends of transcripts as well as splice junctions, expression of desired transcripts can also be quantified, and the nature and extent of alternative splicing can be determined.

As described above, where an adaptor of known sequence is ligated to the 3′ end of target nucleic acids the methods of the present disclosure may find use in generating libraries from both short and long target nucleic acids, both polyadenylated and non-polyadenylated, and/or nucleic acid samples containing various combinations of short, long, polyadenylated and non-polyadenylated target nucleic acids. For example, whether such target nucleic acids are short, long, polyadenylated or non-polyadenylated, the method of the present disclosure may find use in generating libraries that contain the exact sequence(s) of the 3′ ends of the target nucleic acids. For example, because the sequence of the adaptor is known and the adaptors is directly ligated to the 3′ end of the target nucleic acid, the first nucleic acid of the 3′ end may be identified by virtue of being adjacent to the last nucleotide of the known adaptor sequence. Accordingly, methods of the present disclosure may allow for the identification of the exact sequence present at the ends of target nucleic acids in the subject libraries, including where the methods are employed to simultaneously prepare strand specific nucleic acid libraries from combinations of small, large, polyadenylated and non-polyadenylated target nucleic acids, and sub-combinations thereof.

Libraries produced in the subject methods may be produced from a generated product double stranded cDNA. In some embodiments, aspects of the present methods include preparing an expression library. By “expression library” is meant a nucleic acid library useful in evaluating nucleic acid expression of a cellular sample, including e.g., a single cell sample or a sample containing a population of cells. Preparation of expression libraries may include preparing the expression library for next generation sequencing (NGS), including where the NGS expression library is prepared from a RNA sample.

NGS libraries produced as described herein are those whose nucleic acid members include a partial or complete sequencing platform adapter sequence at their termini useful for sequencing using a sequencing platform of interest. Sequencing platforms of interest include, but are not limited to, the HiSeg™, MiSeg™ and Genome Analyzer™ sequencing systems from Illumina®; the Ion PGM™ and Ion Proton™ sequencing systems from Ion Torrent™; Oxford Nanopore™ (e.g., MinION sequencing system), the PACBIO RS II Sequel system from Pacific Biosciences, the SOLiD sequencing systems from Life Technologies™, the 454 GS FLX+ and GS Junior sequencing systems from Roche, the MinION™ system from Oxford Nanopore, or any other sequencing platform of interest.

As described above, the methods of the present disclosure include generating a product double stranded cDNA from a sample that includes RNA. A prepared expression library may be a full length expression library or a non-full length expression library. By “full length expression library” is meant that the nucleic acid members of the library contain either the full length cDNA sequences that correspond to the full length RNA members from which they were reverse transcribed or cDNA of fragments of the full length RNA from which they originated. For example, where an individual library member is a full length cDNA of an mRNA, the full length cDNA will include the entire coding sequence of the mRNA, e.g., the entire spliced mRNA coding sequence, i.e., the entire mRNA coding sequence between the 5′-cap and the poly(A) tail of the mRNA. In some instances a full length expression library will comprise fragments that cover the full length of the original intact RNA transcripts (e.g., in methods that comprise shearing before reverse transcribing, or in methods that comprise random priming along an RNA, i.e., mRNA). A full-length cDNA may or may not include sequence corresponding to one or more untranslated regions (UTR) of an mRNA, e.g., a 3′ UTR or a 5′ UTR. A non-full length expression library can refer to a differential expression library which may comprise sequencing either, or both, the 3′ end or 5′ end of the full length RNA transcript.

A prepared expression library may, in some instances, be a library specifically prepared to capture the ends of the subject RNA molecules. Such libraries may be referred to herein as an “end-captured” library or the members thereof may be referred to as end-captured nucleic acids. End-captured libraries include nucleic acids separately subjected to 3′ end capture or 5′ end capture methods and where the nucleic acids are subjected to both 3′ and 5′ end capture methods. End-capture methods may make use of an end amplification primer. As used herein, the term “end amplification primer” generally refers to a nucleic acid primer used in a PCR reaction to amplify from an end introduced in a double stranded DNA to be amplified. The end introduced into a double stranded DNA to which an end amplification primer binds is generally not an original end of the target nucleic acid or derivative thereof.

Accordingly, in certain embodiments, the methods of preparing expression libraries are end-capture methods. End-capture methods may be employed for sequencing and/or quantifying RNA (e.g., mRNA transcripts), e.g., for differential expression analysis.

In some instances, the subject methods include preparing a plurality of libraries, e.g., a plurality of expression libraries, a plurality of libraries from a plurality of single cells, etc. For example, in some instances, a plurality of individual RNA samples may each be derived from a single cell and the individual RNA samples may be used in the preparation of product double stranded cDNAs and subsequently utilized to produce a plurality of libraries. Where a plurality of libraries is produced, components used in preparing the libraries (e.g., product double stranded cDNAs) or the libraries themselves may or may not be pooled. Where libraries or library preparation components are pooled the nucleic acids may include non-templated identifying nucleic acid sequences that may be utilized in retrospectively identifying the source of a particular library component or sequence thereof. Such retrospective identification may be achieved, e.g., through demultiplexing.

### Compositions and Kits

The present technology also includes various kits and compositions. Useful kits and compositions may include those for use in preparing strand specific nucleic acids, a strand specific nucleic acid library, small RNA libraries, large RNA libraries, small and large RNA libraries and the like. The compositions and kits may include, e.g., one or more of any of the reaction mixture components described above with respect to the subject methods. For example, the compositions and kits may include a nucleic acid sample (e.g., an RNA sample, a combined RNA and DNA sample, etc.), an amplification polymerase (e.g., a thermostable polymerase, etc.), a reverse transcriptase (e.g., a reverse transcriptase capable of template-switching, etc.), a template switch oligonucleotide, dNTPs, a salt, a metal cofactor, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), or any other desired kit component(s).

In some instances, components of the subject compositions and/or kits may be presented as a “cocktail” where, as used herein, a cocktail refers to a collection or combination of two or more different but similar components in a single vessel. Useful cocktails in the subject kits include but are not limited to e.g., “primer cocktails” where the composition of such cocktails may vary and may include e.g., a cocktail of two or more primers.

In certain embodiments, a kit for preparing a strand specific nucleic acid library includes a first adaptor, a primer, and a second adaptor as described herein. The first adaptor can include an adenylated 5′-end and a blocked 3′-end. The primer can include a blocked 5′-end and an extendible 3′-end, where the primer is complementary to a portion of the first adaptor. The second adaptor can include a 3′-homopolymeric portion and a blocked 3′-end. The first adaptor can include at least one cleavable site, such as one or more uracils. The kit can also include uracil-DNA glycosylase to be used in degrading the first adaptor containing one or more uracils as cleavable sites. The first adaptor can include an affinity tag and the kit can further include a binding partner to the affinity tag. The binding partner is coupled to a solid support or a magnetic bead. For example, the affinity tag can be biotin (e.g., the primer is biotinylated) and the binding partner can be streptavidin conjugated to a solid support of a magnetic bead. In this way, any structures hybridized to the primer or any extension products from the primer in the various workflows described herein can be isolated using the binding partner, where unbound materials can be washed away, for example.

The kit can also include a member selected from the group consisting of a kinase, a phosphatase, and combinations thereof (e.g., T4 PNK, rSAP, or other enzymes used in polishing methods, including T4 or Pfu polymerases). Truncated T4 RNA ligase 2 or a mutant form thereof can be included in the kit for use in coupling the first adaptor to the target nucleic acid. An RNA-dependent DNA polymerase, including an RNA-dependent DNA polymerase having terminal transferase activity (e.g., murine leukemia virus reverse transcriptase) can be included in the kit. Various amplification primers can be present, including a first amplification primer complementary to a portion of the second adaptor and a second amplification primer including a portion of the sequence of the first adaptor. At least one of the first amplification primer and the second amplification primer can include a barcode. Various kits can include one or more polymerases, dNTPs, and/or buffers useful for the various reactions operable by one or more enzymes contained in the kit.

In certain instances, the provided kits may include one or more components for performing a template-switching reverse transcription reaction. Such components include but are not limited to those described herein including e.g., a template switching oligonucleotide, a primer, a reverse transcriptase, etc.

In certain embodiments, the kits include reagents for isolating nucleic acids from a nucleic acid source of interest. The reagents may be suitable for isolating nucleic acid samples from a variety of DNA or RNA sources including single cells, cultured cells, tissues, organs, or organisms. The subject kits may include reagents for isolating a nucleic acid sample from a fixed cell, tissue or organ, e.g., formalin-fixed, paraffin-embedded (FFPE) tissue. Such kits may include one or more deparaffinization agents, one or more agents suitable to de-crosslink nucleic acids, and/or the like.

In certain instances, the provided kits may include one or more components for running a plurality of reactions on an automation system (e.g., ICELL8 system from Takara Bio USA). The provided kit can include a multi-well plate (i.e., array chip.) The multi-well array chip can comprise a template switch oligonucleotide and/or any other primer of the disclosure in the wells of the multi-well array chip (e.g., in a dried down format).

Components of the kits may be present in separate containers, or multiple components may be present in a single container.

In addition to the above-mentioned components, a subject kit may further include instructions for using the components of the kit, e.g., to practice the subject methods as described above. In addition, e.g., where the primers and/or oligonucleotides of a kit include a UMI domain, the kit may further include programming for analysis of results including, e.g., decoding encoded UMI domains, counting unique molecular species, etc. The instructions and/or analysis programming are generally recorded on a suitable recording medium. The instructions and/or programming may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The subject compositions may be present in any suitable environment. According to one embodiment, the composition is present in a reaction tube (e.g., a 0.2 mL tube, a 0.6 mL tube, a 1.5 mL tube, or the like) or a well or microfluidic chamber or droplet or other suitable container. In certain aspects, the composition is present in two or more (e.g., a plurality of) reaction tubes or wells (e.g., a plate, such as a 96-well plate, a multi-well plate, e.g., containing about 1000, 5000, or 10,000 or more wells). The tubes and/or plates may be made of any suitable material, e.g., polypropylene, or the like, PDMS, or aluminum. The containers may also be treated to reduce adsorption of nucleic acids to the walls of the container. In certain aspects, the tubes and/or plates in which the composition is present provide for efficient heat transfer to the composition (e.g., when placed in a heat block, water bath, thermocycler, and/or the like), so that the temperature of the composition may be altered within a short period of time, e.g., as necessary for a particular enzymatic reaction to occur. According to certain embodiments, the composition is present in a thin-walled polypropylene tube, or a plate having thin-walled polypropylene wells or materials such as aluminum having high heat conductance. In some instances, the compositions of the disclosure may be present in droplets. In certain embodiments it may be convenient for the reaction to take place on a solid surface or a bead, in such case, the single product nucleic acid primer and/or template switch oligonucleotide, or one or more other primers, may be attached to the solid support or bead by methods known in the art—such as biotin linkage or by covalent linkage—and reaction allowed to proceed on the support. Alternatively, the oligos may be synthesized directly on the solid support—e.g. as described in Macosko, E Z et. al, Cell 161, 1202-1214, May 21, 2015).

Other suitable environments for the subject compositions include, e.g., a microfluidic chip (e.g., a “lab-on-a-chip device”, e.g., a microfluidic device comprising channels and inlets). The composition may be present in an instrument configured to bring the composition to a desired temperature, e.g., a temperature-controlled water bath, heat block, heat block adaptor, or the like. The instrument configured to bring the composition to a desired temperature may be configured to bring the composition to a series of different desired temperatures, each for a suitable period of time (e.g., the instrument may be a thermocycler).

### Nucleic Acids and Analogues Thereof

As described, the present technology includes various nucleic acids, including the particular adaptors and primers and their respective features, which are employed in the various steps of the particular methods outlined herein. These nucleic acids (e.g., adaptors, primers, etc.) can include a nucleic acid or a nucleic acid analogue. The nucleic acid can be formed of ribonucleic acids, deoxyribonucleic acids, or both. Various modified nucleotides can be included in the nucleic acid, such as nucleotides with modified bases, nucleotides with non-natural bases, labeled nucleotides, nucleotides modified with various linkers, and nucleotides conjugated with various chemical moieties, including fluorophores, quenchers, solid supports, and antigenic compounds. The nucleic acid can have one or more labile bases that can be degraded and/or result in strand scission; e.g., deoxy-uracil can be degraded using uracil-DNA glycosylase (UDG) and the abasic site cleaved by subsequent hydrolysis or by apurinic/apyrimidinic (AP) endonuclease. The nucleic acid can have various terminal modifications, including having a phosphorylated or a dephosphorylated 5′-end, the presence or absence of a 3′-end hydroxyl, and can include various terminal blocking groups that prevent modification of the respective terminus by one or more enzyme activities or chemical modifications. For example, the blocking group can prevent ligation at the blocked terminus, polymerase extension at the blocked terminus, or exonuclease activity at the blocked terminus. The nucleic acid can also be formed of a single molecule or can be formed of multiple associated molecules, where the associated molecules are not covalently bonded to each other. For example, multiple oligonucleotides can be hybridized together to form the subject nucleic acid or one or more oligonucleotides can be associated with other molecules through various non-covalent interactions.

By “product nucleic acid” is generally meant a nucleic acid (e.g., a double stranded nucleic acid, such as a dsDNA) containing the complement of a target nucleic acid, including but not limited to e.g., that produced from a reverse transcription reaction. In some instances, a product double stranded cDNA may be produced from a template RNA using a reverse transcription reaction, where any RNA template may be employed including e.g., an mRNA template. Accordingly, the methods provided may include generating a product double stranded cDNA from a template RNA present in an RNA sample through the use of a reverse transcription reaction, such as a template-switching reverse transcription reaction, described in more detail below.

The present technology also provides an adaptor-flanked product formed by any one of the methods described herein. A plurality of adaptor-flanked products can be generated from a plurality of target nucleic acids. The plurality of adaptor-flanked products can constitute a library for subsequent processing steps or analyses, including amplification, sequencing, and various types of qualitative and quantitative analyses.

In certain embodiments, the methods provided further include subjecting a prepared library to an NGS protocol. The protocol may be carried out on any suitable NGS sequencing platform. NGS sequencing platforms of interest include, but are not limited to, a sequencing platform provided by Illumina® (e.g., the HiSeg™, MiSeg™ and/or NextSeg™ sequencing systems); Ion Torrent™ (e.g., the Ion PGM™ and/or Ion Proton™ sequencing systems); Pacific Biosciences (e.g., the PACBIO RS II Sequel sequencing system); Oxford Nanopore™ (e.g., MinION sequencing system), Life Technologies™ (e.g., a SOLiD sequencing system); Roche (e.g., the 454 GS FLX+ and/or GS Junior sequencing systems); or any other sequencing platform of interest. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

In certain embodiments, the subject methods may be used to generate an expression library corresponding to mRNAs for downstream sequencing on a sequencing platform of interest (e.g., a sequencing platform provided by Illumina®, Ion Torrent™, Pacific Biosciences, Life Technologies™, Roche, or the like). According to certain embodiments, the subject methods may be used to generate a NGS library corresponding to non-polyadenylated RNAs for downstream sequencing on a sequencing platform of interest. For example, microRNAs may be polyadenylated and then used as templates in a template switch polymerization reaction as described elsewhere herein. Random or gene-specific priming may also be used, depending on the goal of the researcher. The library may be mixed 50:50 with a control library (e.g., Illumina®'s PhiX control library) and sequenced on the sequencing platform (e.g., an Illumina® sequencing system). The control library sequences may be removed and the remaining sequences mapped to the transcriptome of the source of the mRNAs (e.g., human, mouse, or any other mRNA source).

A prepared expression library may be utilized in various downstream analyses and, in some instances the preparation of the library may be specifically reconfigured for a desired type of downstream analysis. For example, in some instances, a prepared expression library may be subjected to whole transcriptome analysis (WTA) that includes analysis of mRNA as well as non-mRNA RNA species such as non-coding RNA (e.g., snRNA and snoRNA). Therefore, in some instances, library preparation may be specifically configured to allow for analysis of non-mRNA RNAs within the transcriptome, e.g., by utilizing primers that do not rely on hybridization to the poly(A) tail (e.g., random primers) or by the addition of a tailing reaction, e.g., by adding a poly(A) tail to RNA species that are not naturally polyadenylated prior to production of product double stranded cDNA.

### Reaction Conditions

As summarized above, the herein described methods may include certain nucleic acid reactions, including e.g., template-switching reactions, nucleic acid amplification reactions, and the like. The reaction mixture components in such reaction are combined under conditions sufficient to produce the product of the reaction. For example, in some instances, the reaction components of a template-switching reaction are combined under conditions sufficient to produce a product nucleic acid and/or a complex that contains a single product nucleic acid, e.g., hybridized to one or more components. In some instances, the reaction components of a nucleic acid amplification reaction are combined under conditions sufficient to produce an amplified product nucleic acid.

By “conditions sufficient to produce” the subject nucleic acid is meant reaction conditions that permit the relevant nucleic acids and/or other reaction components in the reaction to interact with one another in the desired manner. For example, in some instances, the conditions may be sufficient for nucleic acids of the reaction mixture to hybridize. In some instances, the conditions may be sufficient for an enzyme of the reaction mixture to catalyze a chemical process such as e.g., polymerization, hydrolysis, etc. Achieving suitable reaction conditions may include selecting reaction mixture components, concentrations thereof, and a reaction temperature to create an environment in which the relevant processes proceed, including e.g., the relevant nucleic acids hybridize with one another in a sequence specific manner, the relevant polymerase polymerizes resulting in elongation of a nucleic acid, etc. In addition to specific nucleic acids (e.g., template nucleic acids, oligonucleotides, primers, etc.) of a reaction the reaction mixture may include buffer components that establish an appropriate pH, salt concentration (e.g., KCl concentration), etc. Conditions sufficient to produce a double stranded nucleic acid complex may include those conditions appropriate for hybridization, also referred to as “hybridization conditions”.

Achieving suitable reaction conditions may include selecting reaction mixture components, concentrations thereof, and a reaction temperature to create an environment in which one or more polymerases are active and/or the relevant nucleic acids in the reaction interact (e.g., hybridize) with one another in the desired manner. In suitable reaction conditions, in addition to reaction components, the reaction mixture may include buffer components that establish an appropriate pH, salt concentration (e.g., KCl concentration), metal cofactor concentration (e.g., Mg²⁺ or Mn²⁺ concentration), and the like, for the extension reaction(s) and/or template switching to occur. Other components may be included, such as one or more nuclease inhibitors (e.g., an RNase inhibitor and/or a DNase inhibitor), one or more additives for facilitating amplification/replication of GC rich sequences (e.g., GC-Melt™ reagent (Takara Bio USA, Inc. (Mountain View, Calif.)), betaine, DMSO, ethylene glycol, 1,2-propanediol, or combinations thereof), one or more molecular crowding agents (e.g., polyethylene glycol, or the like), one or more enzyme-stabilizing components (e.g., DTT present at a final concentration ranging from 1 to 10 mM (e.g., 5 mM)), and/or any other reaction mixture components useful for facilitating polymerase-mediated extension reactions and/or template-switching.

One or more reaction mixtures may have a pH suitable for a primer extension reaction and/or template-switching. In certain embodiments, the pH of the reaction mixture ranges from 5 to 9, such as from 7 to 9, including from 8 to 9, e.g., 8 to 8.5. In some instances, the reaction mixture includes a pH adjusting agent. pH adjusting agents of interest include, but are not limited to, sodium hydroxide, hydrochloric acid, phosphoric acid buffer solution, citric acid buffer solution, and the like. For example, the pH of the reaction mixture can be adjusted to the desired range by adding an appropriate amount of the pH adjusting agent.

The temperature range suitable for primer extension reactions may vary according to factors such as the particular polymerase employed, the melting temperatures of any primers employed, etc. In some instances, a reverse transcriptase (e.g., an MMLV reverse transcriptase) may be employed and the reaction mixture conditions sufficient for reverse transcriptase-mediated extension of a hybridized primer include bringing the reaction mixture to a temperature ranging from 4° C. to 72° C., such as from 16° C. to 70° C., e.g., 37° C. to 50° C., such as 40° C. to 45° C., including 42° C.

Nucleic acid reactions, e.g., amplification reactions, of the subject methods may include combining dNTPs into a reaction mixture. In certain aspects, each of the four naturally-occurring dNTPs (dATP, dGTP, dCTP and dTTP) are added to the reaction mixture. For example, dATP, dGTP, dCTP and dTTP may be added to the reaction mixture such that the final concentration of each dNTP is from 0.01 to 100 mM, such as from 0.1 to 10 mM, including 0.5 to 5 mM (e.g., 1 mM). In some instances, one or more types of nucleotide added to the reaction mixture may be a non-naturally occurring nucleotide, e.g., a modified nucleotide having a binding or other moiety (e.g., a fluorescent moiety) attached thereto, a nucleotide analog, or any other type of non-naturally occurring nucleotide that finds use in the subject methods or a downstream application of interest.

Reaction mixtures may be subjected to various temperatures to drive various aspects of the reaction including but not limited to e.g., denaturing/melting of nucleic acids, hybridization/annealing of nucleic acids, polymerase-mediated elongation/extension, etc. Temperatures at which the various processes are performed may be referred to according to the process occurring including e.g., melting temperature, annealing temperature, elongation temperature, etc. The optimal temperatures for such processes will vary, e.g., depending on the polymerase used, depending on characteristics of the nucleic acids, etc. Optimal temperatures for particular polymerases, including reverse transcriptases and amplification polymerases, may be readily obtained from reference texts. Optimal temperatures related to nucleic acids, e.g., annealing and melting temperatures may be readily calculated based on known characteristics of the subject nucleic acid including e.g., overall length, hybridization length, percent G/C content, secondary structure prediction, etc.

According to certain embodiments, the subject methods may include isolating, amplifying and/or analyzing (e.g., sequencing) a deoxyribonucleic acid (DNA). Where the subject methods include isolating, amplifying and/or analyzing DNA the DNA employed may be referred to as a DNA template (or sometimes referred to as template DNA). Template DNAs may be any type of DNA (or sub-type thereof) including, but not limited to, genomic DNA (e.g., animal genomic DNA (e.g., mammalian genomic DNA (e.g., human genomic DNA, rodent genomic DNA (e.g., mouse, rat, etc.), etc.), mitochondrial DNA, or any combination of DNA types thereof or subtypes thereof.

In some instances, methods provided may include isolating and/or purifying a final nucleic acid product (e.g., a nucleic acid library) and/or an intermediate nucleic acid product (e.g., a double stranded product cDNA). Any convenient method of purification may be employed including but not limited to e.g., nucleic acid precipitation (i.e., alcohol precipitation), gel purification, etc.

Several benefits and advantages can be attributed to the present technology as used in the described reactions and methods employing such reactions. One advantage is that the various ways for coupling adaptors to one or more target nucleic acids provided herein can significantly reduce or even prevent the undesired formation of adaptor-dimers or adaptor-multimers, which can hinder further processing and analysis of the adaptor-flanked strand specific nucleic acid library. The present methods can also be faster and easier to perform than other methods that include one or more additional steps to remove or eliminate adaptor-dimers; e.g., size selection by electrophoretic gel purification. The reduction or prevention of adaptor-dimers can also reduce bias and amplification issues for strand specific nucleic acid libraries generated as described herein. By incorporating a cleavable site into the first adaptor, excess first adaptors can be degraded negating the need for intermediate cleanup steps. The template switching aspect of the present technology can also reduce bias in libraries prepared from a population of target nucleic acids using the present methods, further obviating dephosphorylation and rephosphorylation steps necessary in other ligation based methods. In particular, the present technology can significantly reduce ligation-induced bias in preparation of a library of adaptor-flanked products from a sample of target nucleic acids. The present technology may further bypasses the need to decap target nucleic acids that include mRNA, which increases efficiency and reduces the number of steps. These combined advantages further permit a reduction in the input amount of target nucleic acid compared to other methods. An overall savings in time and cost is thereby achieved.

### ILLUSTRATIVE EMBODIMENTS

Embodiments of the present technology are described below in reference to the accompanying figures.

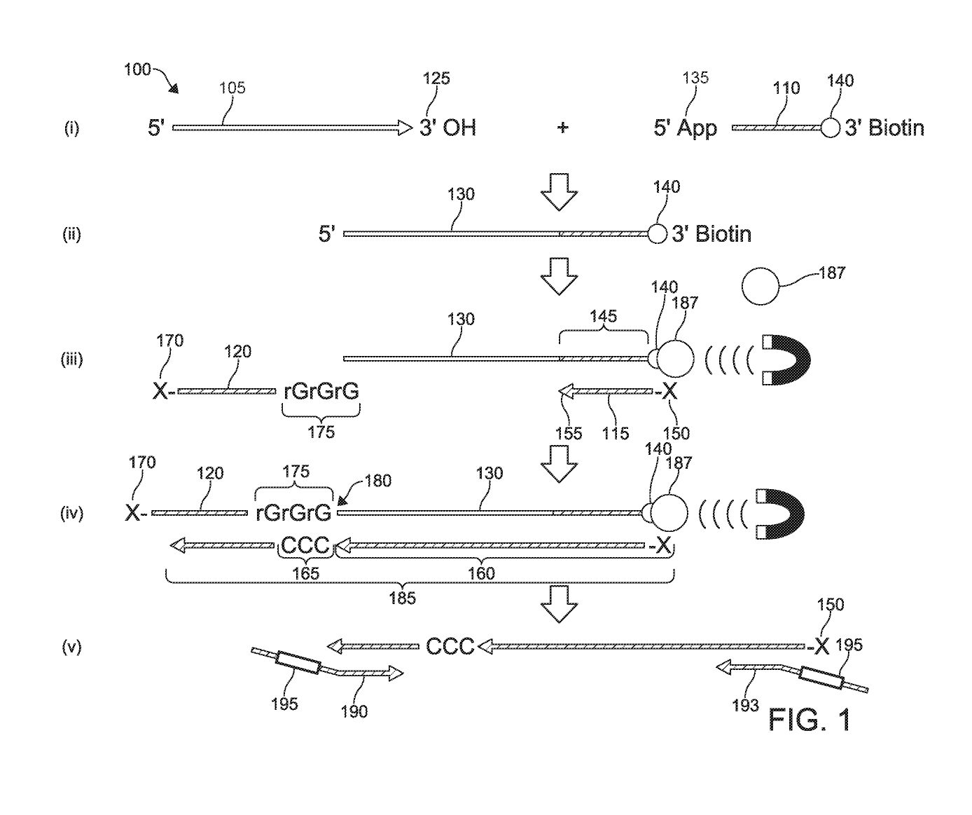

With respect to FIG. 1, a flowchart for a first embodiment of a method 100 of preparing a strand specific nucleic acid library is shown. For ease of reference, the method 100 is shown divided into steps (i)-(v), however, it is understood that aspects of certain steps can occur concomitantly with each other or in a different order and the method 100 is not limited to the particular sequential presentation depicted in FIG. 1. The flowchart, moreover, only depicts a single target nucleic acid 105, a single first adaptor 110, a single primer 115, and a single second adaptor 120, but it is understood that a plurality of target nucleic acids 105, a plurality of first adaptors 110, a plurality of primers 115, and a plurality of second adaptors 120 can be employed, including homogenous or heterogeneous populations of each.

Step (i) shows the combination of the target nucleic acid 105 with the first adaptor 110. The target nucleic acid 105 can include fragmented and repaired total RNA derived from an organism, a tissue, a cell culture, or a single cell. The first adaptor 110 is coupled to a 3′-end 125 of the target nucleic acid 105 to form an adaptor-coupled target nucleic acid 130, as shown in step (ii). For example, the first adaptor 110 can be coupled to a hydroxyl group at the 3′-end 125 of the target nucleic acid 105 by ligation using truncated T4 RNA ligase 2, where the first adaptor 110 includes an adenylated 5′-end 135 and a blocked and biotinylated 3′-end 140. The resulting adaptor-coupled target nucleic acid 130 therefore includes the blocked and biotinylated 3′-end 140 from the first adaptor 110.

The primer 115 is hybridized to the adaptor-coupled target nucleic acid 130 in step (iii). As shown, the primer 115 can be configured to hybridize to a portion 145 of the former first adaptor 110 within the adaptor-coupled target nucleic acid 130. The primer 115 can include a blocked 5′-end 150 and can have an extendible 3′-end 155 that can include a hydroxyl group. A primer extension reaction is performed using a reverse transcriptase to thereby extend the primer 115 and reverse transcribe the adaptor-coupled target nucleic acid 130, including the former target nucleic acid 105, to form a first primer extension product 160 that is complementary to a portion of the adaptor-coupled target nucleic acid 130. One or more non-template directed nucleotides 165 are added to the 3′-end of the first primer extension product 160 by a terminal transferase activity, such as the terminal transferase activity of murine leukemia virus reverse transcriptase, which was also used in extending the primer 115 to form the first primer extension product 160. As shown, three non-template directed cytosines are added to the 3′-end of the first primer extension product 160, but the number of non-template directed nucleotides 165 can vary.

The second adaptor 120 can include a blocked 5′-end 170 and a 3′-end 175 complementary to the one or more non-template directed nucleotides 165 at the 3′-end of the first primer extension product 160. As shown in step (iv), the 3′-end 175 of the second adaptor 120 includes three guanosines complementary to the non-template directed nucleotides 165, but the number of nucleotides complementary to the non-template directed nucleotides 165 can vary. The 3′-end 175 of the second adaptor 120 is hybridized to the non-template directed nucleotides 165 at the 3′-end of the first primer extension product 160, as shown. Accordingly, there is a gap 180 between the second adaptor 120 and the adaptor-coupled target nucleic acid 130; i.e., the second adaptor 120 and the adaptor-coupled target nucleic acid 130 are not coupled together or covalently joined. Base pairing between the non-template directed nucleotides 165 of the first primer extension product 160 and the complementary 3′-end 175 of the second adaptor 120, however, can allow a polymerase with template switching capability to extend the at least one non-template directed nucleotide at the 3′-end of the primer extension product. For example, murine leukemia virus reverse transcriptase can extend the one or more non-template directed nucleotides 165 at the 3′-end of the first primer extension product 160 to form a second primer extension product 185. As can be seen, the second primer extension product 185 is therefore complementary to a portion of the adaptor-coupled target nucleic acid 130 and to a portion of the hybridized second adaptor 120.

The blocked and biotinylated 3′-end 140 of the adaptor-coupled target nucleic acid 130 can be used in one or more ways to isolate various intermediates and products in the method. For example, after the first adaptor 110 is coupled to the 3′-end 125 of the target nucleic acid 105 to form the adaptor-coupled target nucleic acid 130 in steps (i) and (ii), the biotinylated 3′-end 140 of the adaptor-coupled target nucleic acid 130 can be bound to streptavidin conjugated magnetic beads 187. The adaptor-coupled target nucleic acid 130 can therefore be isolated from any uncoupled first adaptor 110, including any uncoupled target nucleic acid 105. Likewise, the blocked and biotinylated 3′-end 140 of the adaptor-coupled target nucleic acid 130 can be used to isolate the first primer extension product 160 base paired therewith, the first primer extension product 160 with one or more non-template directed nucleotides 165 added to the 3′-end thereof, and/or the second primer extension product 185 base paired therewith. Denaturation of the adaptor-coupled target nucleic acid 130 and the second primer extension product 185 can allow further isolation of the adaptor-coupled target nucleic acid 130 bound to the streptavidin conjugated magnetic beads 187 from the second primer extension product 185. These isolation steps can therefore rapidly and conveniently remove unextended primers 115, unhybridized second adaptors 120, and/or other reactants, buffers, enzymes, and aberrant products, and can further include denaturation and release of the second primer extension product 185. In this manner, aberrant reactions and products can be minimized to improve the overall efficiency in preparing the strand specific nucleic acid library.

As shown in step (v), the second primer extension product 185 can be amplified using the polymerase chain reaction and first and second primers 190, 193. The first amplification primer 190 can include a portion of a sequence of the second adaptor 120 and the second amplification primer can include a portion of a sequence of the primer 115 or a portion of a sequence complementary to a portion of the first adaptor 110. Each of the first amplification primer 190 and the second amplification primer 193 includes a barcode 195, which can be the same or different. The resulting amplified strand specific nucleic acid library can be subjected to various analyses, including various next generation sequencing methods.

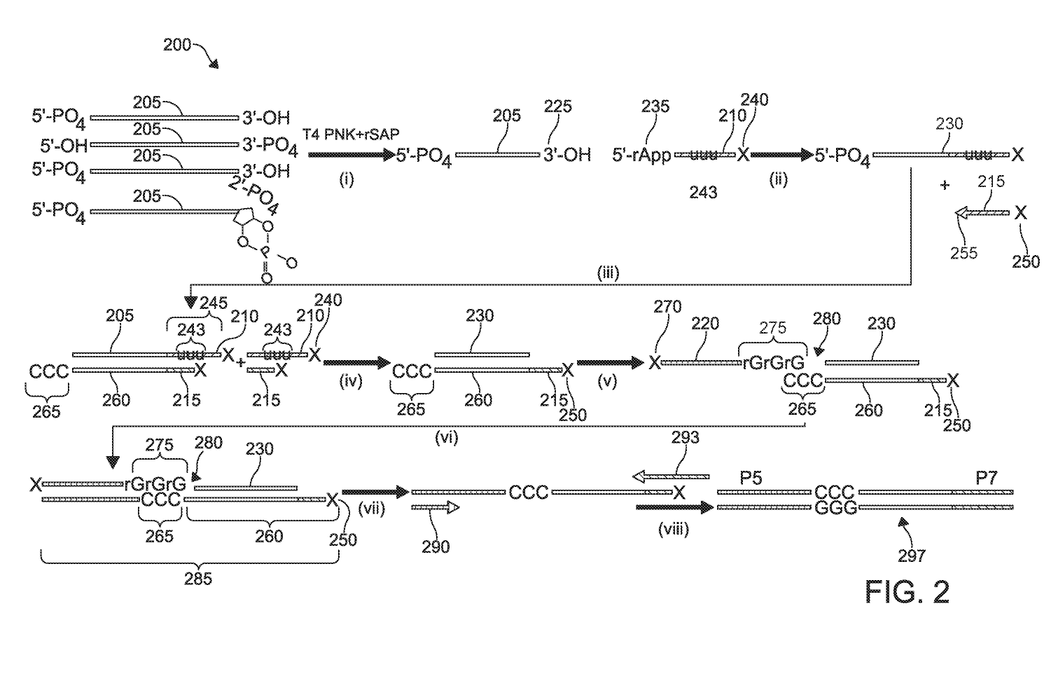

With respect to FIG. 2, a flowchart for a second embodiment of a method 200 of preparing a strand specific nucleic acid library is shown. For ease of reference, the method 200 is shown divided into steps (i)-(viii), however, it is understood that aspects of certain steps can occur concomitantly with each other or in a different order and the method 200 is not limited to the particular sequential presentation depicted in FIG. 2. The flowchart, moreover, only depicts a single target nucleic acid 205, a single first adaptor 210, a single primer 215, and a single second adaptor 220, but it is understood that a plurality of target nucleic acids 205, a plurality of first adaptors 210, a plurality of primers 215, and a plurality of second adaptors 220 can be employed, including homogenous or heterogeneous populations of each.

Various types of target nucleic acid 205 can have various 5′-end and 3′-end terminal structures, including various combinations of 5′ phosphates, 3′ hydroxyls, 5′ hydroxyls, 3′ phosphates, 2′ phosphates and 3′ hydroxyls, and 2′-3′ cyclic phosphates. Such terminal structures can be the result of chemical and/or physical fragmentation of the target nucleic acid 205. Various target nucleic acids 205 can be used in the method 200, including cfDNA, fragmented and denatured dsDNA, genomic DNA, ssDNA, RNA, RNA that is depleted for rRNA and/or enriched for poly(A)+ with oligo(dT), etc. The terminal structures of the target nucleic acid 205 can be repaired or polished using various enzymes, such as T4 polynucleotide kinase (T4 PNK) and recombinant shrimp alkaline phosphatase (rSAP) as shown in step (i), to ensure substantially all of the target nucleic acid 205 includes a hydroxyl group at the 3′-end 225. The repair or polishing of the target nucleic acid 205 can also result in a phosphate group at the 5′-end thereof.

The target nucleic acid 205 is combined with the first adaptor 210 and coupled thereto at step (ii). In particular, the first adaptor 210 is coupled to the hydroxyl group at the 3′-end 225 of the target nucleic acid 205 to form an adaptor-coupled target nucleic acid 230. The first adaptor 210 can be coupled to the target nucleic acid 105 by ligation using a ligase, where the first adaptor 210 includes an adenylated 5′-end 235 and a blocked 3′-end 240. The ligase can include a thermostable 5′AppDNA/RNA ligase and can include a truncated variant of T4 RNA ligase 2. The first adaptor 210 includes one or more cleavable sites 243 therein, which are depicted in FIG. 2 as three U's representing uracils. Various numbers of cleavable sites 243 can be positioned at various locations throughout the first adaptor 210. The resulting adaptor-coupled target nucleic acid 230 therefore includes the blocked 3′-end 240 and the one or more cleavable sites 243 from the first adaptor 210.

The primer 215 is added at step (iii), where the primer 215 can hybridize to the adaptor-coupled target nucleic acid 230 and can hybridize to any remaining uncoupled first adaptor 210. As shown, the primer 215 can be configured to hybridize to a portion 245 of the former first adaptor 210 within the adaptor-coupled target nucleic acid 230, which can also include one or more of the cleavable sites 243. The primer 215 can include a blocked 5′-end 250 and can have an extendible 3′-end 255 that can include a hydroxyl group. A primer extension reaction is performed using a reverse transcriptase to thereby extend the primer 215 and reverse transcribe the adaptor-coupled target nucleic acid 230, including the former target nucleic acid 205, to form a first primer extension product 260 that is complementary to a portion of the adaptor-coupled target nucleic acid 230. One or more non-template directed nucleotides 265 are added to the 3′-end of the first primer extension product 260 by a terminal transferase activity, such as the terminal transferase activity of murine leukemia virus reverse transcriptase, which can also be used in extending the primer 215 to form the first primer extension product 260. As shown, three non-template directed cytosines are added to the 3′-end of the first primer extension product 260, but the number of non-template directed nucleotides 265 can vary.

Step (iv) shows how any remaining uncoupled first adaptor 210, as well as the portion 245 of the former first adaptor in the adaptor-coupled target nucleic acid 230, can be degraded using the one or more cleavable sites 243. Where, as shown in this example, the cleavable sites 243 include one or more uracils, the uracils can be degraded using uracil-DNA glycosylase to form abasic sites, with the resulting abasic sites cleaved by hydrolysis or by use of apurinic/apyrimidinic (AP) endonuclease. The cleavable sites 243 can be positioned throughout the first adaptor 210 to minimize the size of the resulting mono-, di-, and/or oligonucleotide degradation products.

The second adaptor 220 is hybridized to the one or more non-template directed nucleotides 265 at the 3′-end of the first primer extension product. The second adaptor 220 can include a blocked 5′-end 270 and a 3′-end 275 complementary to the one or more non-template directed nucleotides 265 at the 3′-end of the first primer extension product 260. As shown in step (v), the 3′-end 275 of the second adaptor 220 includes three guanosines complementary to the non-template directed nucleotides 265, but the number of nucleotides complementary to the non-template directed nucleotides 265 can vary. The 3′-end 275 of the second adaptor 220 is hybridized to the non-template directed nucleotides 265 at the 3′-end of the first primer extension product 260, as shown. Accordingly, there is a gap 280 between the second adaptor 220 and the adaptor-coupled target nucleic acid 230; i.e., the second adaptor 220 and the adaptor-coupled target nucleic acid 230 are not coupled together or covalently joined. Base pairing between the non-template directed nucleotides 265 of the first primer extension product 260 and the complementary 3′-end 275 of the second adaptor 220, however, can allow a polymerase with template switching capability to extend the at least one non-template directed nucleotide at the 3′-end of the primer extension product. For example, murine leukemia virus reverse transcriptase can extend the one or more non-template directed nucleotides 265 at the 3′-end of the first primer extension product 260 to form a second primer extension product 285 in step (vi). As can be seen, the second primer extension product 285 is therefore complementary to a portion of the adaptor-coupled target nucleic acid 230 and to a portion of the hybridized second adaptor 220.

As shown in step (vii), the second primer extension product 285 can be amplified using the polymerase chain reaction and first and second primers 290, 293. The first amplification primer 290 can include a portion of a sequence of the second adaptor 220 and the second amplification primer can include a portion of a sequence of the primer 215 or a portion of a sequence complementary to a portion of the first adaptor 210. Each of the first amplification primer 290 and the second amplification primer 293 can include a barcode (not shown), as per FIG. 1, where the barcode for each primer can be the same or different. The resulting amplified strand specific nucleic acid library can be subjected to various qualitative and quantitative analyses, including sequencing. In particular, the first amplification primer 290 and the second amplification primer 293 can include sequences required for Next Generation Sequencing (NGS) platforms; e.g., P5 and P7 sequences that bind the flow cell used in the IIlumina Genome Analyzer System. The polymerase chain reaction can be performed using a DNA polymerase, dNTPs, and a buffer that supports PCR amplification. The resulting library of double-stranded DNA products 297 can be amplified to a level suitable for clonal amplification and NGS.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1: Ligation and Template Switching with Degradable Adaptors

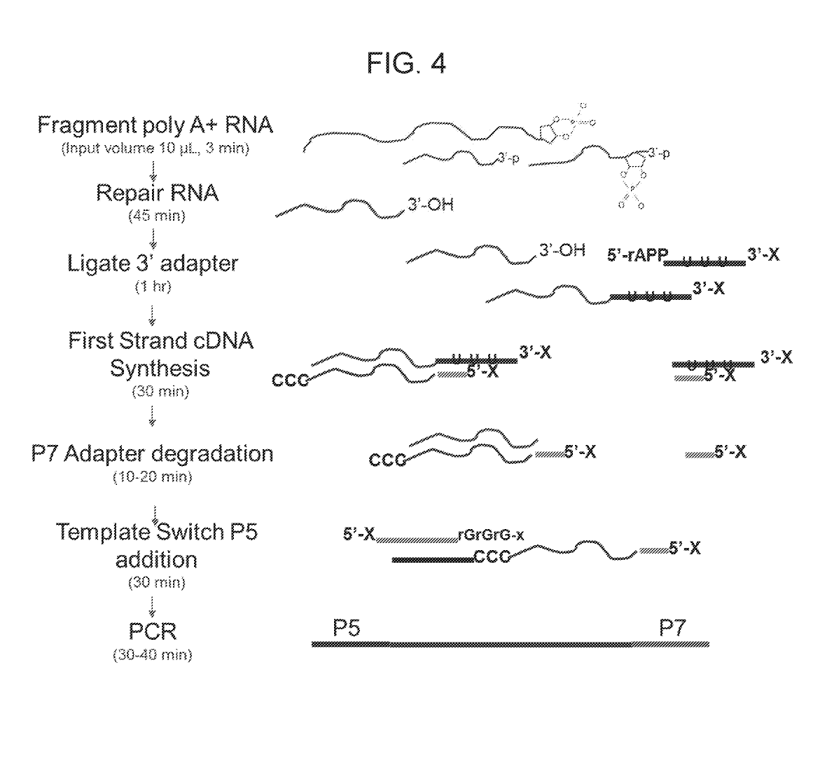

This example describes a method of the present disclosure of using 3′ ligation and template switching with degradable adaptors. In this example and as shown in FIG. 4, the procedure, from RNA to final library preparation, can be completed in 3.5 to 4 hours. RNA-seq libraries are prepared from poly A+ RNA by fragmenting the RNA to 200-300 bases in length via a short incubation at high temperature using a high salt/high pH fragmentation buffer. The fragmented RNA is repaired to make the 3′-ends suitable for ligation. Subsequently, the repaired RNA is ligated to a degradable pre-adenylated 3′-adapter that is blocked at its 3′-end. The ligated RNA is reverse transcribed. Leftover unligated 3′-adapter is degraded using, for example, uracil-deglycosylase which cleaves the uracils located in the adaptor. A 5′-adapter is added to the cDNA samples via reverse transcription by a primer hybridizing to the ligated adaptor, and template switching. The resulting dual adapted cDNA is used in a PCR amplification reaction that incorporates full-length Illumina adapter sequences to the samples, thereby generating a sequencing-ready library.

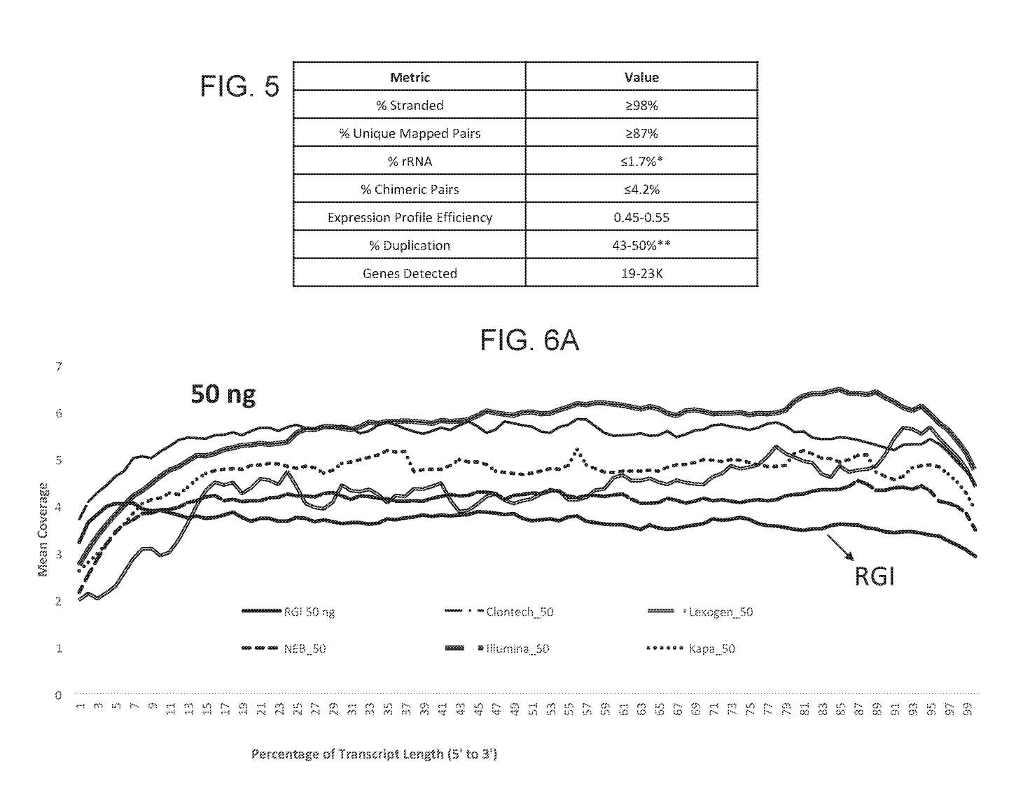

The table in FIG. 5 shows data obtained using the method described in FIG. 4 applied to Human Blood and Peripheral Leukocyte samples. The resulting libraries maintain strandedness, show a high mapping rate to the reference genome used (hg19 in this case), and a good expression profile efficiency (i.e. ratio of reads mapping to exons from the total number of reads). At 1 E+07 reads, the libraries show low mapping to rRNA, lost chimeric reads, and the number of genes detected depended on the input amount of RNA used but was very close between inputs (19-23K genes detected).

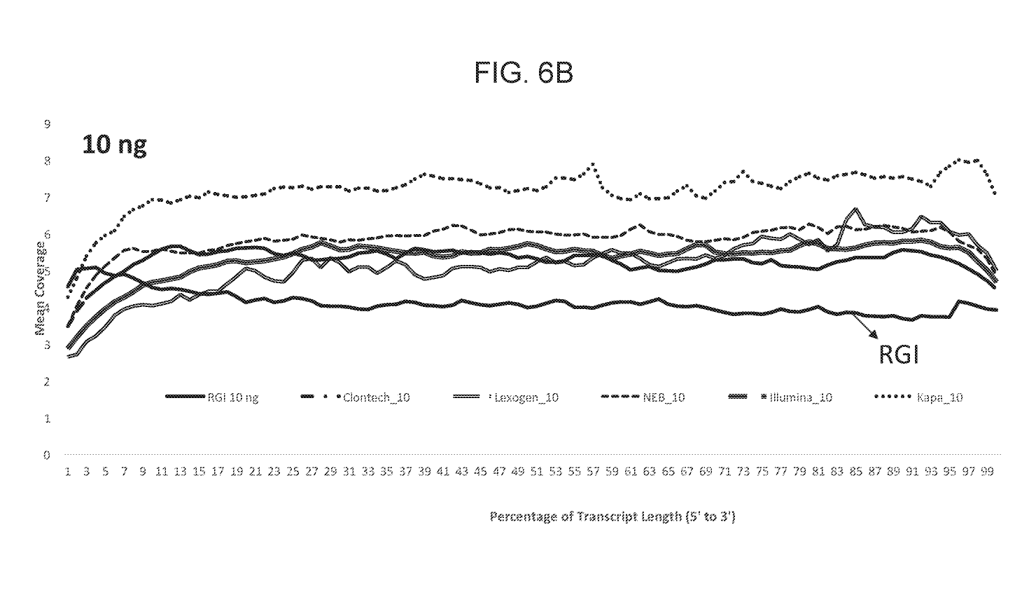

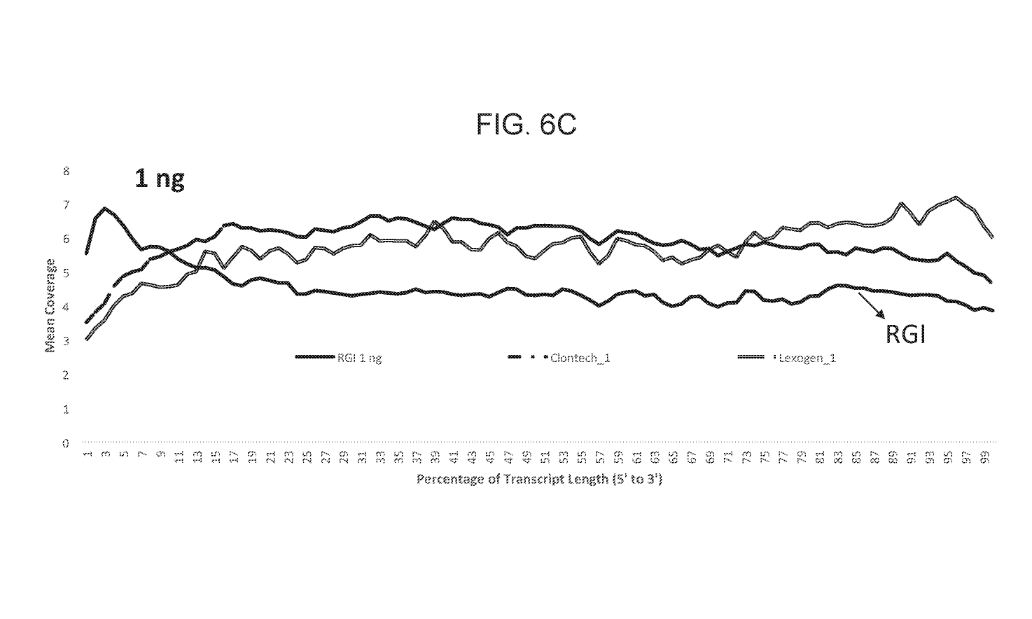

The method produces uniform coverage across low abundance transcripts, as shown in FIG. 6A-6C. Stranded RNA-seq libraries were prepared from 1 or 10 nanograms of Human Blood and Peripheral Leukocyte poly A+ RNA using the 3′-ligation-template switching method as described in FIG. 4 or five different RNA-seq library preparation technologies (Illumina TruSeq Stranded mRNA Sample Prep Kit; Kapa Stranded mRNA-Seq Kit; NEB Ultra Directional RNA Library Prep Kit for Illumina; Lexogen Total RNA-Seq Library Prep Kit). Shown in FIG. 6A-6C are average coverage plots across the length of three transcripts determined by the RNA-SeQC pipeline to have the highest expression levels in the lowest expressed transcripts, for all technologies analyzed. The 3′-ligation-template switching method shows more even coverage compared to the other technologies tested and had less bias at the 5′ and 3′-ends of the transcripts.

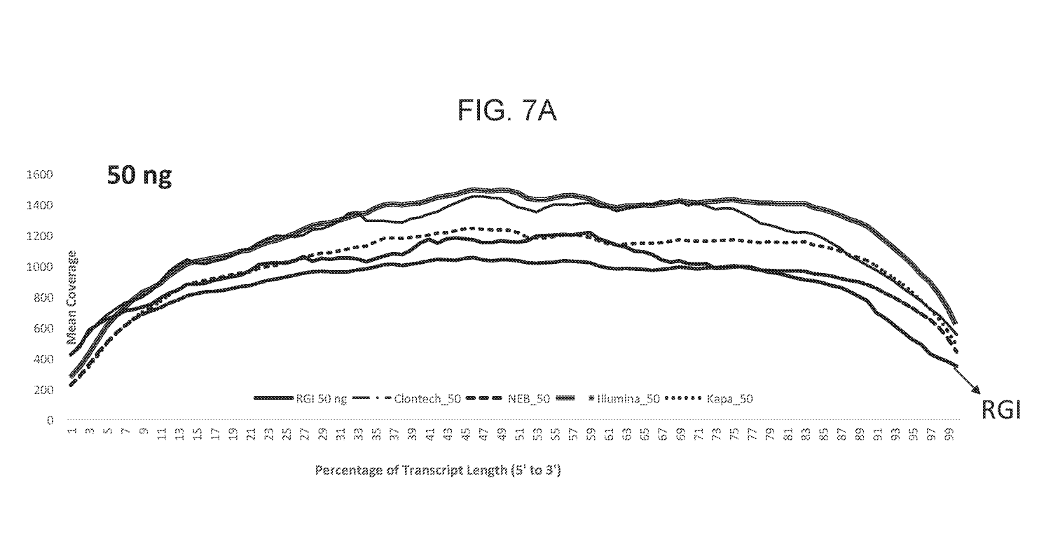

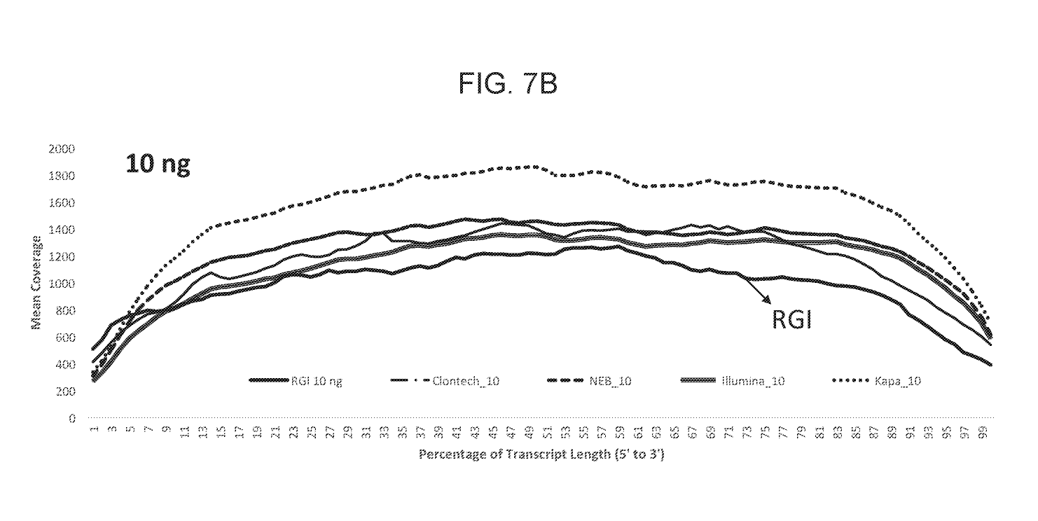

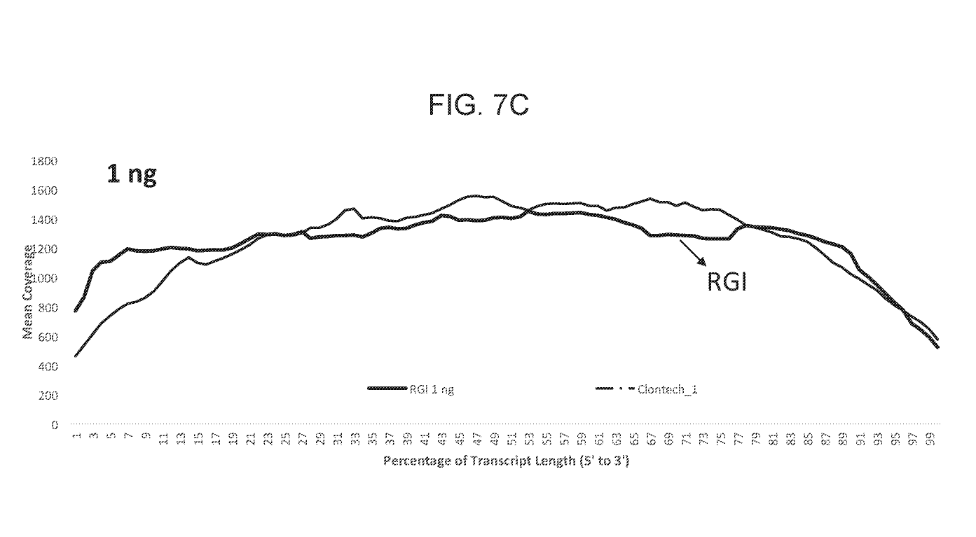

FIG. 7A-7C display that the 3′ ligation and template switching method also shows uniform of coverage across high abundance transcripts. Shown are average coverage plots across the length of three transcripts determined by the RNA-SeQC pipeline to have the highest expression levels in the highly expressed transcripts, for all technologies analyzed. The method of the disclosure shows more even coverage as compared to all other technologies tested, with less bias at the 5′ and 3′-ends of the transcripts.

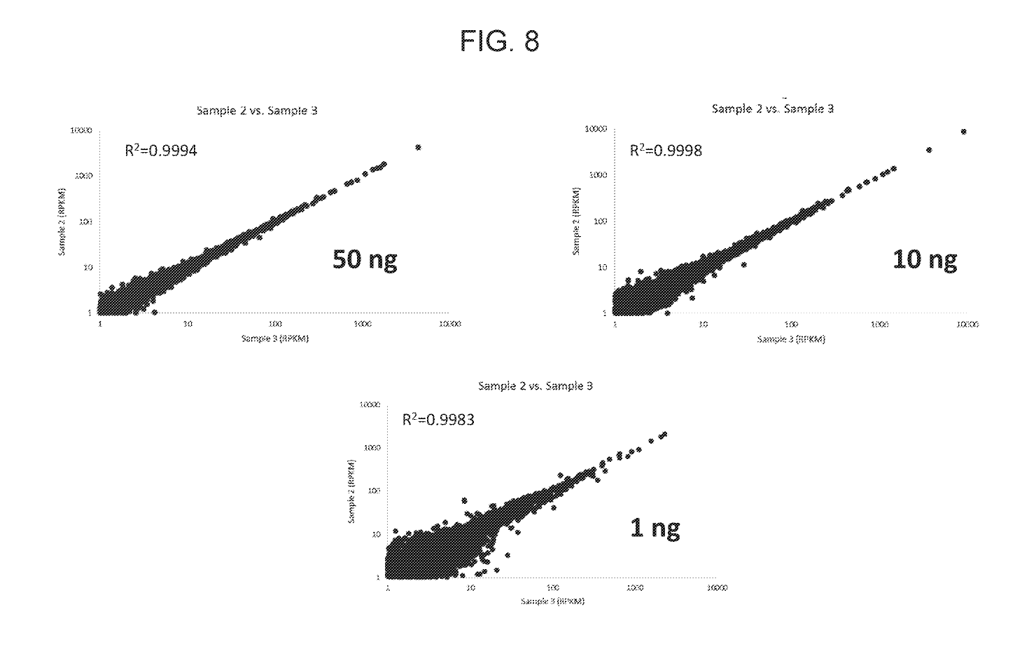

The 3′ ligation and template switching method as described above was tested for technical reproducibility as shown in FIG. 8. Technical replicates from libraries prepared from 50, 10 or 1 ng mRNA input amounts (Clontech Takara Human Peripheral Leukocyte poly A+ RNA) show strong concordance (R²≥0.99). Transcript abundances were normalized to Reads per Kilobase of transcript per Million mapped reads. The method was highly reproducible across various input amounts.

### Example 2: Comparison of 3′ Ligation and Template Switching with polyA Tailing

The method of 3′ ligation and template switching as described herein, was tested using 1 ng of human brain total RNA (Fisher Scientific, Cat no AM7962) or 1 ng of miRXplore Universal Reference (Miltenyi Biotech Inc., Cat. No. 130-093-521). The 5′ pre-adenylated adaptor, comprising at least a portion of Illumina Read Primer 1, was ligated onto the 3′ end of RNA molecules with T4 RNA ligase buffer and RNA Ligase 2 truncated KQ (NEB M0373). The ligation reaction was incubated at 25° C. for 6 hrs followed by 4° C. for 16 hrs.

Following ligation, reverse transcription was initiated by annealing a DNA oligo (e.g., reverse transcription oligonucleotide primer) complementary to the ligated adapter. The DNA oligonucleotide was annealed by incubation for 2 min at 72° C., followed by 4° C. for 3 min, followed by addition of First Strand Buffer, RNase Inhibitor, dNTPs, DTT, a template switching oligonucleotide comprising at least a portion of Illumina Read Primer 2, and PrimeScript reverse transcriptase, and incubated at 42° C. for 1 h. The reaction was terminated by a 10 min incubation at 72° C.

Following reverse transcription, 40% of the cDNA was used for addition of full-length Illumina adapters by PCR. The cDNA was diluted five time into the PCR reaction consisting of SeqAmp DNA Polymerase, SeqAmp PCR buffer, and PCR primers comprising full length Illumina flow cell adapters, then subjected to PCR amplification (1 min at 98° C., then 12 cycles (for miRXplore template RNA) or 16 cycles (for human brain RNA), each cycle comprising 10 sec at 98° C., 5 sec at 60° C., and 10 sec at 68° C. The PCR products were purified using AMPure XP beads at a 1.8:1 beads:sample ratio.

For sequencing, the PCR products were size selected using the BluePippin Size Selection System (Sage Science, BLU0001) and 3% Agarose Gel Cassettes (Sage Science, BDF3010) to target the size expected for miRNAs. Libraries were sequenced on a MiSeq instrument, performing single end reads with 50 cycles. Sequencing reads for all libraries were trimmed and annotated using CLC Genomics Workbench 9.5.1 (Qiagen) and Small RNA Analysis tools, allowing no more than one mismatch during mapping. For the human brain RNA sample, miRNA sequences were mapped to miRBase (release 21).

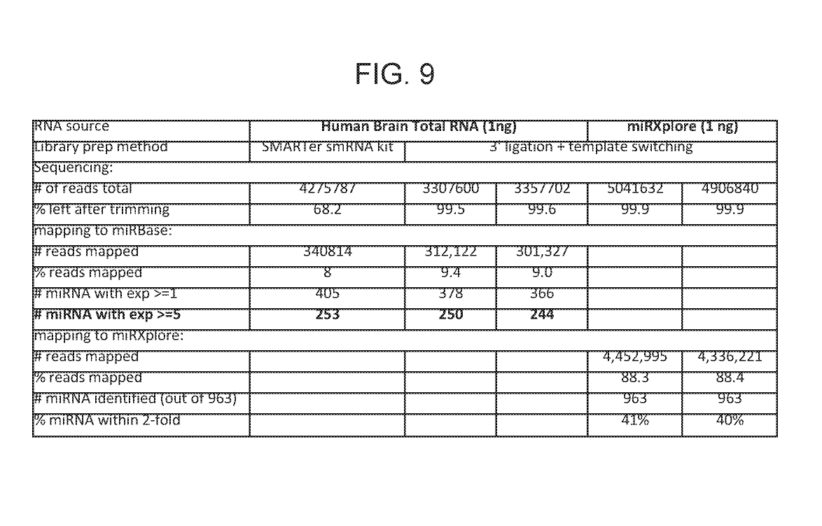

For analysis of the miRXplore libraries, reads were annotated using a reference file containing sequences present in the synthetic pool. Normalization was performed by determining, for each miRNA, the ratio between the observed number of reads and the predicted number of reads. All 963 miRNAs present in the miRXplore library were identified and 40% were found to be within 2-fold of the expected expression level, as shown in FIG. 9.

Compared to the Smarter smRNA kit which utilizes polyA tailing of miRNAs followed by oligo-dT primer binding, reverse, transcription, and template switching, the 3′ ligation and template switching methods resulted in reduced adaptor dimer formation as evidenced by the high number of percent reads after trimming.

### Example 3: Method of Simultaneously Preparing a Small and Large RNA Sequencing Library

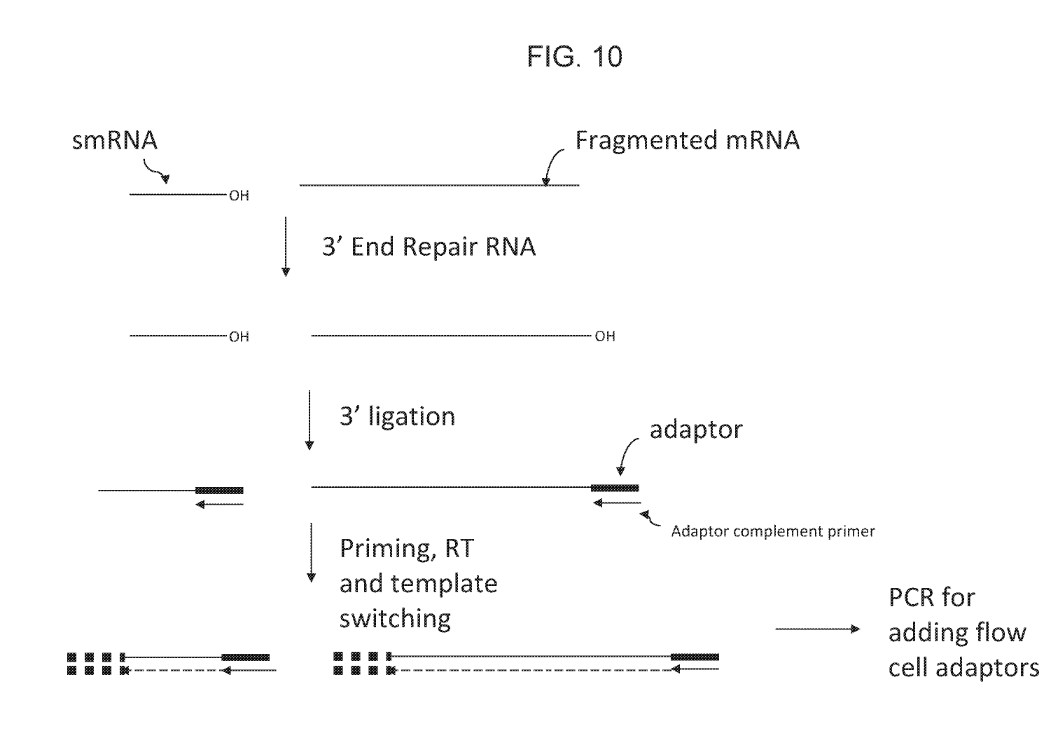

This example describes a method of preparing a smRNA library and a large RNA library from the same sample, as shown in FIG. 10. Total RNA comprising smRNA and fragmented mRNA is 3′ end repaired to add a hydroxyl to the ends of the smRNA and mRNA. An adaptor of the disclosure is ligated to the 3′ end of the smRNA and mRNA. A primer that is capable of hybridizing to a sequence in the ligated adaptor is hybridized to the adaptor and incubated with reaction mixture components to allow reverse transcription and template switching. The sample is amplified with PCR primers comprising sequencing flow cell adaptors, thereby generating a sequencing-ready library comprising both smRNA and large mRNA.

### Example 4: Method of Simultaneously Preparing a Small and Large RNA Sequencing Library Through Random Priming or Oligo-Dt Priming

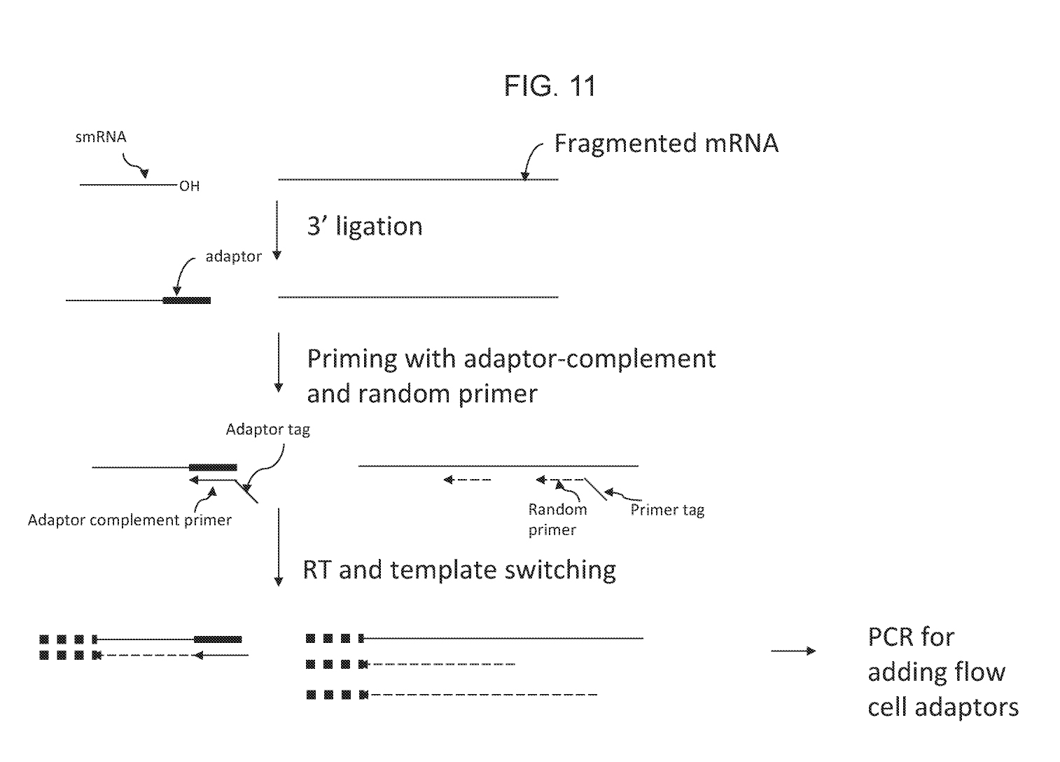

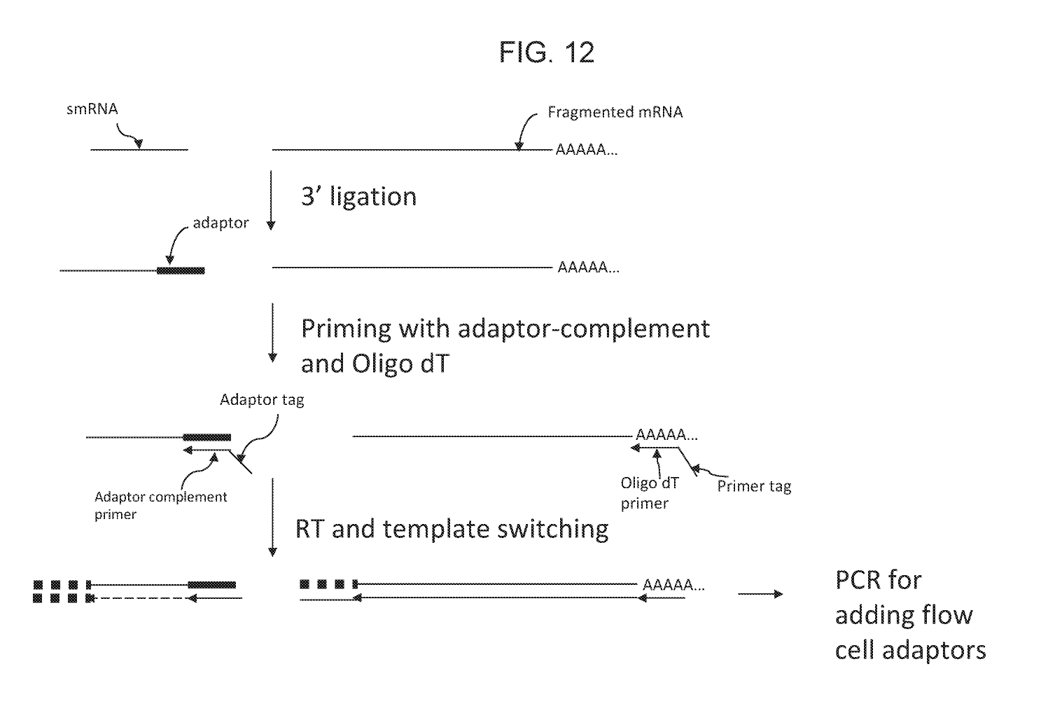

This example describes a method of preparing a smRNA library and a large RNA library from the same sample using random priming or oligo-dt priming, and 3′ ligation, as shown in FIG. 11 and FIG. 12, respectively. Total RNA comprising smRNA and fragmented mRNA is provided. An adaptor of the disclosure is ligated to the 3′ end of the smRNA. A primer that is capable of hybridizing to a sequence in the ligated adaptor is hybridized to the adaptor and incubated with reaction mixture components to allow reverse transcription and template switching. The primer comprises an adaptor tag sequence, which is an arbitrary sequence specific to the adaptor. Reads comprising the adaptor tag sequence are determined to originate from a target RNA that was reverse transcribed by a primer that hybridized to a ligated adaptor (e.g., small RNA).

In the same tube, a random primer is hybridized to the fragmented mRNA and allowed to reverse transcribe and template switch. The random primer comprises a primer tag sequence, which is an arbitrary sequence specific to the random primers. Reads comprising the primer tag sequence are determined to originate from a target RNA that was reverse transcribed by a random primer (e.g., large RNA).

The sample is amplified with PCR primers comprising sequencing flow cell adaptors, thereby generating a sequencing-ready library comprising both smRNA and large mRNA. Through the use of two different primers with two different tags, it is possible to distinguish the sequences that were captured by random priming versus adaptor ligation. In this way, sequencing reads can be separated for small and large RNAs.

Notwithstanding the appended claims, the disclosure is also defined by the following clauses:

1. A method comprising:

coupling an adaptor to a 3′-end of a target nucleic acid to form an adaptor-coupled target nucleic acid comprising an adaptor domain and a target nucleic acid domain;

combining:
the coupled target nucleic acid;
a primer comprising a 3′ domain that hybridizes to at least a portion of the adaptor domain;
a template switch oligonucleotide;
a polymerase; and
dNTPs,

into a reaction mixture under conditions sufficient to produce a complex comprising the coupled target nucleic acid and the template switch oligonucleotide each hybridized to a single product nucleic acid polymerized from the dNTPs in a template switching reaction.

2. The method according to Clause 1, wherein the target nucleic acid is:

a small target nucleic acid of 100 nucleotides or less in length; or

a large target nucleic acid of 150 nucleotides or greater in length, present in a mixture comprising a plurality of small target nucleic acids of 100 nucleotides or less in length and a plurality of large target nucleic acids of 150 nucleotides or greater in length.

3. The method according to Clauses 1 or 2, wherein the target nucleic acid is a polyadenylated nucleic acid or a non-polyadenylated nucleic acid in a mixture comprising both polyadenylated and a non-polyadenylated nucleic acids.
4. The method according to any of the preceding clauses, further comprising:

fragmenting the target nucleic acid prior to the coupling to generate target nucleic acid fragments;

end-repairing the nucleic acid fragments to produce at least one end-repaired target nucleic acid fragment; and

coupling the end-repaired target nucleic acid fragment to the adaptor to form an adaptor-coupled end-repaired target nucleic acid.

5. The method according to any of the preceding clauses, wherein the adaptor comprises an adenylated 5′-end and a blocked 3′-end prior to the coupling, and coupling the adaptor to the 3′-end of the target nucleic acid comprises ligating the adenylated 5′-end of the adaptor to a 3′-hydroxyl group of the target nucleic acid.
6. The method according to any of the preceding clauses, further comprising degrading the adaptor, the template switch oligonucleotide or both.
7. The method according to Clause 6, wherein the adaptor, the template switch oligonucleotide or both comprise at least one cleavable site and the degrading comprises cleaving the at least one cleavable site.
8. The method according to Clause 7, wherein the cleavable site comprises a uracil.
9. The method according to any of the preceding clauses, wherein the polymerase is an RNA-dependent DNA polymerase having terminal transferase activity.
10. The method according to any of the preceding clauses, further comprising amplifying single product nucleic acid.
11. The method according to Clause 10, wherein the amplifying comprising contacting the single product nucleic acid with a first amplification primer comprising at least a portion of a sequence present in the primer or the adaptor domain and a second amplification primer comprising at least a portion of a sequence present in the template switch oligonucleotide.
12. The method according to any of the preceding clauses, wherein at least one of the primer, the adaptor, the template switch oligonucleotide, the first amplification primer, the second amplification primer or a combination thereof includes a barcode.
13. A strand specific nucleic acid library generated according to the method of Clause 1.
14. The library according to Clause 13, wherein the library comprises:

a first single product nucleic acid, or amplification product thereof, comprising sequence of a small target nucleic acid of 100 nucleotides or less in length; and

a second single product nucleic acid, or amplification product thereof, comprising sequence of a large target nucleic acid of 150 nucleotides or greater in length.

15. A kit for preparing a strand specific nucleic acid library, the kit comprising:

an adaptor including an adenylated 5′-end and a blocked 3′-end;

a template switch oligonucleotide; and

one or more ligation components sufficient to couple the adaptor to the 3′-end of a target nucleic acid.

16. A method of preparing a strand specific nucleic acid library, the method comprising:

coupling a first adaptor to a 3′-end of a target nucleic acid to form an adaptor coupled target nucleic acid;

hybridizing a primer to the adaptor coupled target nucleic acid;

extending the primer to form a first primer extension product complementary to a portion of the adaptor coupled target nucleic acid;

adding at least one non-template directed nucleotide to a 3′-end of the first primer extension product;

hybridizing a portion of a second adaptor to the at least one non-template directed nucleotide at the 3′-end of the first primer extension product;

extending the at least one non-template directed nucleotide at the 3′-end of the primer extension product to form a second primer extension product complementary to a portion of the adaptor coupled target nucleic acid and to a portion of the hybridized second adaptor.

17. The method of Clause 16, wherein the first adaptor includes an adenylated 5′-end and a blocked 3′-end, and coupling the first adaptor to the 3′-end of the target nucleic acid includes ligating the adenylated 5′-end of the first adaptor to a 3′-hydroxyl group of the target nucleic acid.
18. The method of Clause 17, wherein the 3′-hydroxyl group of the target nucleic acid is formed by reacting the target nucleic acid with a member selected from the group consisting of a kinase, a phosphatase, and combinations thereof.
19. The method of any of Clauses 16 to 18, wherein the first adaptor includes at least one cleavable site.
20. The method of Clause 19, wherein the at least one cleavable site comprises uracil.
21. The method of Clauses 19 or 20, further comprising cleaving the at least one cleavable site in the first adaptor after extending the primer to form a first primer extension product complementary to a portion of the adaptor coupled target nucleic acid.
22. The method of any of Clauses 16 to 21, wherein the first adaptor includes a barcode.
23. The method of any of Clauses 16 to 22, wherein the second adaptor includes a barcode.
24. The method of any of Clauses 16 to 23, wherein the target nucleic acid comprises RNA.
25. The method of any of Clause 16 to 23, wherein the target nucleic acid comprises single-stranded DNA.
26. The method of any of Clauses 16 to 25, wherein the primer includes a blocked 5′-end and an extendible 3′-end.
27. The method of any of Clauses 16 to 26, wherein the first adaptor includes an affinity tag.
28. The method of Clause 27, further comprising isolating the adaptor coupled target nucleic acid using the affinity tag.
29. The method of Clause 28, wherein isolating the adaptor coupled target nucleic acid using the affinity tag includes isolating the adaptor coupled target nucleic acid from uncoupled first adaptor and any uncoupled target nucleic acid.
30. The method of any of Clauses 16 to 29, wherein extending the primer to form the first primer extension product complementary to the portion of the adaptor coupled target nucleic acid is performed using an RNA-dependent DNA polymerase.
31. The method of any of Clauses 16 to 30, wherein adding at least one non-template directed nucleotide to the 3′-end of the first primer extension product is performed using an RNA-dependent DNA polymerase having terminal transferase activity.
32. The method of Clause 31, wherein the RNA-dependent DNA polymerase having terminal transferase activity comprises murine leukemia virus reverse transcriptase.
33. The method of any of Clauses 16 to 32, wherein the second adaptor includes a blocked 5′-end and/or a blocked 3′-end, the blocked 3′-end having complementarity to the at least one non-template directed nucleotide at the 3′-end of the primer extension product.
34. The method of any of Clauses 16 to 33, wherein extending the at least one non-template directed nucleotide at the 3′-end of the primer extension product to form the second primer extension product is performed using an RNA-dependent DNA polymerase capable of template switching.
35. The method of Clause 34, wherein the RNA-dependent DNA polymerase capable of template switching comprises murine leukemia virus reverse transcriptase.
36. The method of any of Clauses 16 to 35, further comprising amplifying the second primer extension product.
37. The method of Clause 36, further comprising sequencing the amplified second primer extension product.
38. The method of Clause 37, wherein amplifying the second primer extension product includes performing a polymerase chain reaction using a first amplification primer including a portion of a sequence of the second adaptor and a second amplification primer including a portion of a sequence of the primer or a portion of a sequence complementary to a portion of the first adaptor.
39. The method of Clause 38, wherein at least one of the first amplification primer and the second amplification primer includes a barcode.
40. The method of Clause 39, further comprising sequencing the amplified second primer extension product.
41. The method according to any of Clauses 16 to 40, wherein the second adaptor comprises a template switch oligonucleotide.
42. The method of Clause 41, wherein the second adaptor further comprises a barcode.
43. A strand specific nucleic acid library formed by the method of any of Clauses 16 to 42.
44. A kit for preparing a strand specific nucleic acid library, the kit comprising:

a first adaptor including an adenylated 5′-end and a blocked 3′-end;

a primer including a blocked 5′-end and an extendible 3′-end, the primer complementary to a portion of the first adaptor; and

a second adaptor including a 3′-homopolymeric portion and a blocked 3′-end.

45. The kit of Clause 44, wherein the first adaptor includes at least one cleavable site.
46. The kit of Clause 45, wherein the at least one cleavable site comprises uracil.
47. The kit of Clause 46, further comprising uracil-DNA glycosylase.
48. The kit of any of Clauses 44 to 47, wherein the first adaptor includes a barcode.
49. The kit of any of Clauses 44 to 48, wherein the second adaptor includes a barcode.
50. The kit of any of Clauses 44 to 49, wherein the first adaptor includes an affinity tag.
51. The kit of Clause 50, further comprising a binding partner to the affinity tag.
52. The kit of Clause 51, wherein the binding partner is coupled to a solid support or a magnetic bead.
53. The kit of any of Clauses 44 to 52, further comprising a member selected from the group consisting of a kinase, a phosphatase, and combinations thereof.
54. The kit of any of Clauses 44 to 53, further comprising a truncated T4 RNA ligase 2 or a mutant form thereof.
55. The kit of any of Clauses 44 to 54, further comprising an RNA-dependent DNA polymerase.
56. The kit of Clause 55, wherein the RNA-dependent DNA polymerase has terminal transferase activity.
57. The kit of any of Clauses 44 to 56, further comprising:

a first amplification primer complementary to a portion of the second adaptor; and

a second amplification primer including a portion of the sequence of the first adaptor.

58. The kit of Clause 57, wherein at least one of the first amplification primer and the second amplification primer includes a barcode.
59. The kit of any of Clauses 44 to 58, further comprising a polymerase.
60. The kit of any of Clauses 44 to 59, further comprising dNTPs.
61. A population of adaptor coupled nucleic acids comprising:

a target nucleic acid comprising a 3′ adaptor;

a first primer extension product hybridized to the target nucleic acid comprising at least one non-template directed nucleotide on the 3′ end of the first primer extension product; and

a second adaptor hybridized to at least a portion of the non-template directed nucleotide.

62. The population according to Clause 61, wherein the target nucleic acid comprises DNA.
63. The population according to Clause 61, wherein the target nucleic acid comprises RNA.
64. The population according to Clause 62, wherein the target nucleic acid comprises ssDNA.
65. The population according to Clause 64, wherein the target nucleic acid comprises cell-free DNA.
66. The population according to Clause 64, wherein the target nucleic acid comprises DNA from FFPE tissue.
67. The population according to any of Clauses 61 to 66, wherein the 3′ adaptors in the population each comprise a barcode.
68. The population according to Clause 67, wherein each adaptor comprises the same barcode.
69. The population according to Clause 68, wherein the barcode is a sample barcode used to determine the identity of the sample from which the target nucleic acid originated.
70. The population according to any of Clauses 61 to 69, wherein the barcode for each 3′ adaptor in the population is different.
71. The population according to Clause 70, wherein the barcode comprises a unique molecular identifier and is used to determine expression levels of the target nucleic acid.
72. The population according to any of Clauses 61 to 71, wherein the 3′ adaptor is attached to a solid support.
73. The population according to any of Clauses 61 to 72, wherein the second adaptor comprises a modification on its 5′ end selected from the group consisting of: an abasic lesion, a nucleotide adduct, iso-nucleotide base, and combinations thereof.
74. The population of any of Clauses 61 to 72, wherein the second adaptor comprises a modified nucleotide selected from the group consisting of: LNA, FANA, 2′-O-Me RNA, 2′-fluoro RNA, and combinations thereof.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method comprising:
coupling an adaptor to a 3′-end of a target nucleic acid to form an adaptor-coupled target nucleic acid comprising an adaptor domain and a target nucleic acid domain;
combining:
the coupled target nucleic acid;
a primer comprising a 3′ domain that hybridizes to at least a portion of the adaptor domain;
a template switch oligonucleotide;
a polymerase; and
dNTPs,
into a reaction mixture under conditions sufficient to produce a complex comprising the coupled target nucleic acid and the template switch oligonucleotide each hybridized to a single product nucleic acid polymerized from the dNTPs in a template switching reaction.

2. The method according to claim 1, wherein the target nucleic acid is:
a small target nucleic acid of 100 nucleotides or less in length; or
a large target nucleic acid of 150 nucleotides or greater in length,
present in a mixture comprising a plurality of small target nucleic acids of 100 nucleotides or less in length and a plurality of large target nucleic acids of 150 nucleotides or greater in length.

3. The method according to claim 1, wherein the target nucleic acid is a polyadenylated nucleic acid or a non-polyadenylated nucleic acid in a mixture comprising both polyadenylated and a non-polyadenylated nucleic acids.

4. The method according claim 1, further comprising:
fragmenting the target nucleic acid prior to the coupling to generate target nucleic acid fragments;
end-repairing the nucleic acid fragments to produce at least one end-repaired target nucleic acid fragment; and
coupling the end-repaired target nucleic acid fragment to the adaptor to form an adaptor-coupled end-repaired target nucleic acid.

5. The method according claim 1, wherein the adaptor comprises an adenylated 5′-end and a blocked 3′-end prior to the coupling, and coupling the adaptor to the 3′-end of the target nucleic acid comprises ligating the adenylated 5′-end of the adaptor to a 3′-hydroxyl group of the target nucleic acid.

6. The method according claim 1, further comprising degrading the adaptor, the template switch oligonucleotide or both.

7. The method according to claim 6, wherein the adaptor, the template switch oligonucleotide or both comprise at least one cleavable site and the degrading comprises cleaving the at least one cleavable site.

8. The method according to claim 7, wherein the cleavable site comprises a uracil.

9. The method according claim 1, wherein the polymerase is an RNA-dependent DNA polymerase having terminal transferase activity.

10. The method according claim 1, further comprising amplifying single product nucleic acid.

11. The method according to claim 10, wherein the amplifying comprising contacting the single product nucleic acid with a first amplification primer comprising at least a portion of a sequence present in the primer or the adaptor domain and a second amplification primer comprising at least a portion of a sequence present in the template switch oligonucleotide.

12. The method according claim 1, wherein at least one of the primer, the adaptor, the template switch oligonucleotide, the first amplification primer, the second amplification primer or a combination thereof includes a barcode.

13. A strand specific nucleic acid library generated according to the method of claim 1.

14. The library according to claim 13, wherein the library comprises:
a first single product nucleic acid, or amplification product thereof, comprising sequence of a small target nucleic acid of 100 nucleotides or less in length; and
a second single product nucleic acid, or amplification product thereof, comprising sequence of a large target nucleic acid of 150 nucleotides or greater in length.

15. A kit for preparing a strand specific nucleic acid library, the kit comprising:
an adaptor including an adenylated 5′-end and a blocked 3′-end;
a template switch oligonucleotide; and
one or more ligation components sufficient to couple the adaptor to the 3′-end of a target nucleic acid.

16. The kit according to claim 15, wherein the adaptor includes at least one cleavable site.

17. The kit according to claim 16, wherein the at least one cleavable site comprises uracil.

18. The kit according to claim 15, further comprising uracil-DNA glycosylase.

19. The kit according to claim 15, wherein the adaptor includes a barcode.

20. The kit according to claim 15, further comprising a member selected from the group consisting of a kinase, a phosphatase, and combinations thereof.
